(19) United States
(12) Patent Application Publication
(10) Pub. No.: US 2023/0103330 A1
(43) Pub. Date: Apr. 6, 2023

(54) MECHANISTIC BIOMARKER FOR PREDICTING THE SURVIVAL OF PANCREATIC CANCER PATIENTS

(71) Applicant: The Regents of the University of California, Oakland, CA (US)

(72) Inventors: Alexandra C NEWTON, San Diego, CA (US); Timothy R BAFFI, San Diego, CA (US); Gordon B MILLS, Portland, OR (US)

(73) Assignee: The Regents of The University of California, Oakland, CA (US)

(21) Appl. No.: 17/909,908

(22) PCT Filed: Mar. 18, 2021
(86) PCT No.: PCT/US2021/022926

§ 371 Date: Sep. 7, 2022

Related U.S. Application Data
(60) Provisional application No. 62/992,379, filed on Mar. 20, 2020.

(51) Int. Cl. G01N 33/574

(52) U.S. Cl. CPC G01N 33/57438; G01N 33/57484; G01N 2333/912; G01N 2333/916

(57) ABSTRACT

The present disclosure provides biomarkers and methods of use thereof for diagnosing and prognosing pancreatic cancer and other cancers in a subject. The biomarkers comprise protein kinase C (PKC), PH domain and leucine rich repeat protein phosphatase 1 (PHLPP1), and the ratio of PKC/PHLPP1 and can be detected using anti-PKC and/or anti-PHLPP1 antibodies and quantified using immunochemistry techniques. Methods of treating pancreatic cancer with PHLPP1 inhibitors are also provided.

### CROSS REFERENCE TO RELATED APPLICATIONS

This application claims the benefit of U.S. Provisional Application No. 62/992,379, filed on Mar. 20, 2020, which is incorporated herein by reference in its entirety.

### STATEMENT OF GOVERNMENT INTEREST

This invention was made with government support under grant Nos. CA217842 and GM122523, both awarded by National Institutes of Health (NIH). The government has certain rights in the invention.

### CROSS REFERENCE TO SEQUENCE LISTING

The genetic components described herein are referred to by sequence identifier numbers (SEQ ID NO). The SEQ ID NOs correspond numerically to the sequence identifiers <400>1, <400>2, etc. The sequence listing in written computer readable format (CRF) submitted Mar. 18, 2021, as a text file named “942103-2040_Sequence_Listing_ST25.txt created on Mar. 18, 2021, and having a size of 1,302 bytes, is incorporated by reference in its entirety.

### FIELD OF INVENTION

The present disclosure relates generally to biomarkers for diagnosing and/or prognosing pancreatic cancer.

### BACKGROUND OF INVENTION

Cellular homeostasis depends on exquisite regulation of protein kinase and phosphatase activity to allow precise responses to extracellular signals (Brognard and Hunter, 2011). Deregulation of phosphorylation mechanisms is a hallmark of disease, with aberrant kinase and phosphatase activity driving an abundance of pathologies. One kinase family whose activity must be precisely tuned to avoid pathophysiologies is protein kinase C (PKC). PKC family members transduce myriad signals downstream of phospholipid hydrolysis to regulate diverse cellular functions such as proliferation, apoptosis, migration, and differentiation (Griner and Kazanietz, 2007; Newton, 2018). Although assumed to be oncoproteins for decades, analysis of cancer-associated mutations and protein-expression levels supports a general tumor-suppressive role for PKC isozymes, accounting for the failure and, in some cases, worsened patient outcome of PKC inhibitors in cancer clinical trials (Antal et al., 2015b; Zhang et al., 2015). Conversely, enhanced PKC activity is associated with degenerative diseases, such as Alzheimer's disease and spinocerebellar ataxia, and with increased risk of cerebral infarction (Newton, 2018). Even small changes in PKC activity drive pathogenesis, as illustrated by a germline mutation in affected family members with Alzheimer's disease that causes a modest 30% increase in the catalytic rate of the enzyme (Callender et al., 2018). Thus, tight regulation of PKC signaling output is essential.

PKC isozymes are multi-domain Ser/Thr kinases whose activity is governed by reversible release of an autoinhibitory pseudosubstrate segment (Newton, 2018). For conventional PKC (cPKC) isozymes (FIG. 1A; a, b, and g), this is controlled by binding of the lipid second messenger diacylglycerol (DAG) to the second of two tandem C1 domains. Ca²+binding to a plasma membrane-directing C2 domain facilitates activation of these isozymes by localizing them on the membrane, thereby increasing the probability of binding DAG. Engaging both the C2 and C1B domains on membranes provides the energy to release the pseudosubstrate, allowing substrate phosphorylation and downstream signaling. This activation is short-lived, with the enzyme reverting to the autoinhibited conformation upon return of Ca²+ and DAG to unstimulated levels.

Like many kinases, PKC is also regulated by phosphorylation. However, unlike many kinases, these phosphorylations occur shortly after biosynthesis and are constitutive (Bomer et al., 1989; Keranen et al., 1995). Newly synthesized cPKC is matured by phosphorylation at three conserved positions: the activation loop by the phosphoinositide-dependent kinase PDK-1 (Dutil et al., 1998; Le Good et al., 1998) and two C-terminal sites, the turn and hydrophobic motifs (Keranen et al., 1995). The C-tail phosphorylations depend upon both the kinase complex mammalian target of rapamycin complex 2 (mTORC2) (Guertin et al., 2006) and the intrinsic catalytic activity of PKC, with in vitro studies showing that PKC autophosphorylates by an intramolecular reaction at the hydrophobic motif (Behn-Krappa and Newton, 1999).

Mechanisms that prevent the phosphorylation of PKC, such as loss of PDK-1, inhibition ofmTORC2, orimpairmentof PKC's intrinsic catalytic activity, result in PKC degradation (Balendran etal., 2000; Guertinetal., 2006; Hansraetal., 1999). Indeed, it is this sensitivity of the unphosphorylated species to degradation that accounts for the ability of phorbol esters, potent PKC agonists, to cause the “downregulation” of PKC (Jaken et al., 1981). The membrane-engaged active conformation of PKC is highly sensitive to dephosphorylation (Dutil et al., 1994), and dephosphorylation at the hydrophobic motif by the Pleckstrin homology (PH) domain leucine-rich repeat protein phosphatase (PHLPP) serves as the first step in the degradation of PKC, triggering subsequent PP2A-dependent dephosphorylation at the turn motif and activation loop (Gao et al., 2008; Hansra et al., 1999; Lu et al., 1998). Thus, PKC signaling output is regulated not only by second messengers but also by mechanisms that establish the level of PKC protein in the cell. Understanding how to modulate these levels has important therapeutic implications, as high PKC levels correlate with improved survival in diverse cancers (Newton, 2018).

### SUMMARY OF THE INVENTION

The present disclosure provides a quality control mechanism in which PHLPP1 ensures the fidelity of PKC maturation by proofreading the conformation of newly-synthesized PKC. Specifically, phosphorylation of the hydrophobic motif is necessary to adopt an autoinhibited conformation, and this autoinhibited conformation then protects the hydrophobic motif from dephosphorylation by PHLPP1, thus protecting PKC from degradation. In cancer, hotspot mutations in the pseudosubstrate are loss-of-function (LOF) because of this proofreading mechanism. The ratio of hydrophobic motif phosphorylation to total PKCa in over 5,000 tumor samples reveals a near 1:1 ratio, validating mechanistic studies showing that if PKC is not phosphorylated at the hydrophobic motif, it is degraded. High levels of PKC hydrophobic motif phosphorylation (and hence total PKC) correlate inversely with PHLPP1 levels and co-segregate with improved patient survival in pancreatic adenocarcinoma, implicating PKC phosphorylation as both a prognostic marker and therapeutic target. This PHLPP1-dependent quality control mechanism provides a general LOF mechanism for a tumor suppressor in cancer by targeting post-translational modifications.

In pancreatic cancer, the amount of PHLPP1 is the dominant mechanism controlling PKC levels. The present disclosure provides that pancreatic cancer patients can be stratified for treatment and survival based on the protein levels of PKC and PHLPP1. High PKC levels and low PHLPP1 levels have protective effects for pancreatic adenocarcinoma. Accordingly, the present disclosure provides that identifying pancreatic cancer patients with low PKC levels and high PHLPP1 levels is an excellent way to stratify patients and treat this particular subset with PHLPP1 inhibitors.

In terms of assays for pancreatic cancer patients, fine needle biopsies and core needle biopsies are standard today; genomic-based tests are also standard of care in pancreatic cancer and IHC (immunohistochemistry) can be used to evaluate microsatellite instability. For these reasons, performing IHC using antibodies to PKC and PHLPP1 can be easily added to current tests.

Therefore, the present disclosure provides biomarkers for diagnosing and/or prognosing pancreatic cancer patients, the biomarkers comprising PKC and PHLPP1, wherein PKC level is high and PHLPP1 level is low, indicating protective effects for pancreatic cancer. In certain embodiments, the ratio of PKC protein to PHLPP1 protein in patient tumor samples provides a mechanistic biomarker for the prognostic stratification of pancreatic adenocarincoma patients based on survival rates. Methods for diagnosing and/or prognosing pancreatic cancer patients with anti-PKC antibodies and/or anti-PHLPP1 antibodies are also provided. There is currently no metric available for predicting the survival of pancreatic cancer patients or standard for stratification. The present disclosure provides a mechanistic, protein-level metric that can be measured in a sensitive, high-throughput assay to dramatically stratify patients by 5-year survival rates (-40% survival vs 0% survival in one TCGA pancreatic adenocarcinoma patient cohort).

Furthermore, the present disclosure provides a method of treating pancreatic cancer of a patient comprising administering to said patient a PHLPP1 inhibitor, and measuring a ratio of PKC/PHLPP1 to determine protective effects.

### BRIEF DESCRIPTION OF THE DRAWINGS

FIGS. 1A-1E. PKC Priming Phosphorylations Are Necessary for Maturation and Activity. FIG. 1A. cPKC domain structure showing pseudosubstrate, C1 domains, C2 domain, kinase domain, C-terminal tail, and three priming phosphorylations (circles). FIG. 1B. Crystal structure of PKCβII kinase domain (PDB: 3PFQ) highlighting priming phosphorylations (space filling) and pseudosubstrate residues 18-26 modeled into the active site. FIG. 1C. COS7 cells expressing CKAR alone (endogenous) or co-expressing the indicated mCherry-PKCβII WT or Ala mutant constructs were treated with PDBu (200 nM) followed by G66983 (1 mM). FIG. 1D. Immunoblot (IB) analysis of COS7 cells transfected with the indicated PKCβII constructs and probed with indicated phospho-specific or total PKC antibodies. FIG. 1E. COS7 cells expressing CKAR alone (endogenous) or co-expressing the indicated mCherry-PKCβII phosphomimetic Glu substitution constructs were treated with PDBu (200 nM) followed by G66983 (1 mM). CKAR data represent the normalized FRET ratio changes (mean±SEM) from at least 3 independent experiments of >100 cells for each condition.

FIGS. 2A-2G. The Autoinhibitory Pseudosubstrate Is Required for Cellular PKC Phosphorylation. FIG. 2A. Schematic of pseudosubstrate-deleted PKC (DPS) lacking amino acids 19-6 of PKCa or PKCβII. FIG. 2B. IB analysis of lysates from COS7 cells transfected with indicated PKC constructs and probed with phospho-specific or total PKC antibodies. (FIGS. 2C-2G) PKC activity analysis in COS7 cells expressing CKAR alone or co-expressing the indicated mCherry-PKC constructs treated with G66976 (1 mM) (FIG. 2C), agonists UTP (100 mM), PDBu (200 nM), and inhibitors BisIV (2m M) (FIG. 2D) and G66983 (1 mM) (FIG. 2E), or G66976 (1 mM) (FIG. 2F and FIG. 2G). PKCβII DPS trace in (FIG. 2F) is reproduced in (FIG. 2G) for comparison (dashed line). CKAR data represent the normalized FRET ratio changes (mean±SEM) from three independent experiments of >100 cells for each condition.

FIGS. 3A-3J. The Autoinhibited Conformation of PKC Retains Priming Phosphorylations. FIG. 3A. Schematic showing PKC conformations assessed using the Kinameleon C FRET reporter. FIG. 3B. PKCβII pseudosubstrate mutants (SEQ ID Nos: 1-3) with basic and neutral residues highlighted. Crystal structure of PKCβII showing the pseudosubstrate modeled into the active site of the kinase domain with basic residues shown as sticks is shown. The pseudo-P-site is indicated by an asterisk (*). FIG. 3C. Absolute FRET ratio (mean±SEM) of the indicated PKCβII Kinameleon C constructs expressed in COS7 cells. Each data point represents the average absolute FRET ratio from an individual cell. FIG. 3D. FRET ratio changes (mean±SEM) of the indicated PKCβII Kinameleon C constructs expressed in COS7 cells following PDBu (200 nM) treatment. FIG. 3E. Representative YFP images of indicated PKCβII Kinameleon C constructs in COS7 cells before (basal) or after (post-PDBu) 25 min stimulation with PDBu (200 nM). FIG. 3F. Representative images of plasma membrane-targeted (PM) or Golgi-targeted (Golgi) mCFP and the indicated mCherry-PKCβII in COS7 cells. Co-localization is shown in an overlay of mCFP and mCherry images (Merge). FIG. 3G. Schematic for PKC Translocation Assay: agonist-stimulated movement of mYFP-tagged PKC to plasma membrane-localized myristoylated-palmitoylated mCFP is monitored by FRET increase upon PKC membrane association. FIG. 3H. Translocation analysis of the indicated mYFP-PKCβII was monitored by FRET ratio change in COS7 cells co-expressing myristoylated-palmitoylated mCFP and treated with PDBu (100 nM). FIG. 3I. Basal PKC activity in COS7 cells expressing CKAR and the indicated mCherry-PKC@ll constructs treated with G66983 (1 mM). Quantification (right) shows the normalized magnitude of FRET ratio change. FIG. 3J. IB analysis of lysates of COS7 cells expressing indicated PKC@ll constructs and probed with indicated phospho-specific or total PKC antibodies. ****p<0.0001, by repeated-measures one-way ANOVA and Brown-Forsythe Test; n.s., not significant. Kinameleon and CKAR represent the normalized FRET ratio changes (mean±SEM) from three independent experiments with >100 cells for each condition.

FIGS. 4A-4K. Autoinhibition Protects PKC from PHLPPI-Mediated Dephosphorylation and Degradation. FIG. 4A. Schematic of indicated PKCpil truncation mutants. FIG. 4B. IB analysis of COS7 cells expressing indicated PKC@ll constructs probed with indicated phospho-specific or total PKC antibodies.

FIG. 4C. Autoradiogram (MS) and IB analysis of newly synthesized PKC pulse-chase immunoprecipitates from COS7 cells expressing HA-PKCpil and FLAG-PHLPP1. FIG. 4D. IB analysis of FLAG immunoprecipitates from COS7 cells transfected with indicated HA-PKCpil and FLAG-PHLPP1 constructs and probed with indicated antibodies. Vinculin was used as a loading control. FIG. 4E. IB analysis of lysates from COS7 cells transfected with indicated HA-PKCpil constructs probed with phospho-specific or total PKC antibodies. PKC@ll A37-86 (AC1A), PKC@llIA159-291 (AC2), PKC@llIA101-291 (AC1B/C2), PKC@llIA37-291 (AC1A/C1B/C2), or PKCpil 296-673 (Cat). Quantification (bottom) of pSerm⁰ band intensity relative to WT (mean±range, n=2) is shown. FIG. 4F. COS7 cells co-expressing CKAR and indicated mCherry-PKCpil regulatory domain deletion constructs were treated with BislV (2 pM). Insert shows trace for Cat activity, with cluster of traces in main figure reproduced for comparison. Quantification (bottom) shows magnitude of the FRET ratio change from 3 independent experiments. FIG. 4G. Autoradiogram (MS) of HA immunoprecipitates from pulse chase of COS7 cells expressing the indicated PKC constructs. FIG. 4H. IB analysis of lysates from Sf9 insect cells infected with the indicated GST-PKC constructs and His-PHLPP1 PP2C (PHLPP1; 1,154-1,422) baculovirus, probed with the indicated phospho-specific or total PKC antibodies. Quantification (right) of total PKC protein normalized to Tubulin or PKC phosphorylation normalized to total PKC is shown. FIG. 4I. IB analysis of lysates from COS7 cells expressing the indicated HA-PKC constructs treated with cycloheximide (CHX, 250 pM) for the indicated times prior to lysis and probed with the indicated antibodies. Quantification (bottom) of PKC band intensity normalized to Tubulin loading control and plotted as percentage of protein at time zero is shown. FIG. 4J. IB analysis of lysates from WT (Phlpp1+*) or PHLPP1 knockout (Phlpp1 4) MEFs treated with PDBu (200 nM) for the indicated time points prior to lysis and probed with the indicated antibodies. Quantification (bottom) of PKC band intensity normalized to Hsp90 loading control and plotted as percentage of protein at time zero is shown. FIG. 4K. IB analysis of lysates from untreated WT (Phlpp1+/+) or PHLPP1 knockout (Phlpp1 4) MEFs probed with the indicated antibodies. Quantification (bottom) of PKC band intensity normalized to Tubulin loading control is shown. *p<0.05, **p<0.01, ***p<0.001, ****p<0.0001 by repeated-measures one-way ANOVA and Tukey HSD test. IB quantification (excluding E) represent the mean±SEM from at least three independent experiments. Dashed line (B and E) indicates splicing of irrelevant lanes from a single blot.

FIGS. 5A-5F. Cancer-Associated Pseudosubstrate Hotspot Mutations Reveal a Distinct PKC LOF Mechanism. FIG. 5A. Mutations in the N terminus (1-40) of PKCβ (SEQ ID NO: 4) identified in human cancers showing 3D-clustered functional hotspots. FIG. 5B. Crystal structure of PKCpil with the pseudosubstrate modeled into the active site. Interactions between the pseudosubstrate (Arg22), bound nucleotide (ATP), and kinase domain (Asp4⁷⁰) are highlighted. KinView analysis showing evolutionary protein sequence conservation of the activation segment from all PKC isozymes (top, SEQ ID NO: 5) or all protein kinases (bottom) is shown. Height of the letter indicates the residue frequency at that position. FIG. 5C. COS7 cells co-expressing CKAR and indicated mCherry-PKC@ll cancer-associated pseudosubstrate mutants were treated with G66983 (1 mM) to determine basal activity. FIG. 5D. Quantification of FIG. 5C. showing the magnitude of FRET ratio change upon inhibitor addition. Data represent three independent experiments of >100 cells for each construct; dotted line indicates WT activity; ****p<0.0001, by repeated-measures one-way ANOVA and Tukey-Kramer HSD test. FIG. 5E. IB analysis of lysates from COS7 cells expressing indicated PKC constructs probed with phospho-specific or total PKC antibodies. FIG. 5F. Schematic of cancer-associated pseudosubstrate mutants. Pseudosubstrate mutations manifest as LOF, either by enhancing (reduced activity) or disrupting (reduced stability) PKC autoinhibition.

FIGS. 6A-6B. PKC Quality Control Is Conserved in Human Cancer. FIG. 6A. RPPA analysis of TCGA Pan Cancer Atlas tumor samples showing hydrophobic motif phosphorylation and total protein levels for PKC, Akt, or S6K. Cancer types are indicated by TCGA study abbreviations. FIG. 6B. Quantification of FIG. 6A: scatterplot of the expression of the indicated phosphorylation correlated with the associated total protein.

FIGS. 7A-7E. High PKC and Low PHLPPI Levels Are Protective in Pancreatic Adenocarcinoma. FIG. 7A. Heatmap of PKCa expression by cancer type (Expression; column 1). Heatmap showing coefficient of determination between PKCa hydrophobic motif phosphorylation and the indicated protein or phosphorylation (correlation; columns 2-8) is shown. Cancer types are indicated by TCGA study abbreviations. FIG. 7B. RPPA analysis of PHLPP1 and PKCa levels in patient samples from the indicated cancers with Least-Squares Regression Line. FIG. 7C. Heatmap of individual PAAD patients showing relative abundance of the indicated protein or modification. FIG. 7D. Kaplan-Meier survival plots from PAAD patients stratified by PKC hydrophobic motif phosphorylation levels. p=log-rank p value. FIG. 7E. Model of PKC Quality Control by PHLPP1: newly synthesized PKC (i) binds PHLPP1 where it surveys the conformation of this unprimed PKC to regulate phosphorylation of the hydrophobic motif. This species is in an open conformation, with the pseudosubstrate (PS; rectangle) and all membrane-targeting modules unmasked. PKC that becomes phosphorylated (ii) is immediately autoinhibited, releasing PHLPP1 and entering the pool of stable, catalytically competent but inactive enzyme (iii). This primed species is transiently and reversibly activated by binding second messengers (iv). PKC that does not properly autoinhibit following priming phosphorylations (v), for example due to mutations that impair autoinhibition, is rapidly dephosphorylated by PHLPP1 at the hydrophobic motif, leading to further dephosphorylation and degradation (vi).

FIG. 8. PKC APS Phosphorylation Is Not Regulated by a Calyculin-Sensitive Phosphatase. Western blot of lysates from COS-7 cells expressing RFP-tagged PKCβII wild-type (WT) or deleted pseudosubstrate (APS) and treated with DMSO (−) or Calyculin A (Cal, 100pM) and Go6976 (6pM) for 20 minutes prior to lysis. Immunoblots were probed with PKC phospho-specific antibodies against the activation loop (pThr⁵⁰⁰), turn motif (pThr” ¹), or hydrophobic motif (pSer⁸O), total overexpressed PKC (HA), or phospho-serine substrate (pSer) as a positive control for Calyculin A.

FIG. 9. Substrate Specificity of PKC Phosphorylation Site Mutants. Western blot of lysates from COS-7 cells expressing mCherry-tagged PKCβII wild-type (WT), or PKCβII mutants PKCβII T500V, PKCβII T641A, PKCβII S660A, PKCβII ΔPS, PKCβII ΔPS 641A, PKCβII ΔPS 660A, or mCherry vector control (Vec) stimulated (PDBu) with DMSO (−) or 200 nM PDBu (+) for 3 min prior to lysis and pretreated (BisV/83) with either DMSO (−) or 1pM BislV/1pM Go6983 (+) 10 min prior to PDBu addition. Immunoblots were probed for total PKCβ , phospho-Ser PKC substrate antibodies, and Tubulin loading control.

FIG. 10. Table of Cancer-Associated PKCβII Pseudosubstrate Mutations. Mutations identified from patient-derived tumor samples showing the resultant missense mutation in PKCβ , cancer type, sample identifier, and database source.

FIGS. 11A-11B. PKC is Fully Phosphorylated at the Hydrophobic Motif in Human Cancer Cell Lines. FIG. 11A. Heatmap of kinase levels versus their hydrophobic motif phosphorylation for PKC or Akt1 obtained from 5,157 patient samples from TCGA Pan Cancer Atlas measured by Reverse Phase Protein Array (RPPA). Shown are total PKCa versus pSer⁸⁵⁷ and total Akt1 versus pSer⁴⁷³ and pThr³⁰⁸. FIG. 11B. Quantification of data in (A): Scatterplot of the expression of the indicated phosphorylations correlated with PKCa or Akt1 protein levels; R=Spearman's rank correlation coefficient.

FIG. 12. Graphical summary. PKC generally functions as a tumor suppressor. A quality control mechanism in which PHLPP1 opposes priming phosphorylation of newly synthesized PKC to suppress its steady-state levels is discovered. This quality control dominates in pancreatic cancer: patients with high levels of PHLPP1 and low levels of PKC have worsened survival.

### DETAILED DESCRIPTION OF THE INVENTION

The present disclosure provides biomarkers for diagnosing and/or prognosing pancreatic cancer patients comprising PKC, PHLPP1, and a ratio of PKC/PHLPP1, wherein PKC level is high and PHLPP1 level is low indicating protective effects for pancreatic cancer. Methods for diagnosing and/or prognosing pancreatic cancer patients with anti-PKC antibodies and/or anti-PHLPP1 antibodies are also provided. Furthermore, the present disclosure provides method of treating pancreatic effects comprising administering PHLPP1 inhibitors.

Compositions and methods for treating disease associated with PHLPP are disclosed in WO 2006105490, the entire content of which is incorporated by reference herewith.

In certain embodiments, the present disclosure provides that Phosphorylation of newly synthesized PKC is necessary for stabilizing autoinhibition; PHLPP1 dephosphorylates newly synthesized PKC to provide quality control PKC quality control. The present disclosure provides a PKC “quality control” mechanism, in which the phosphatase PHLPP1 negatively regulates the levels of PKC by removing a stabilizing phosphorylation that is essential for PKC expression (>0.99 correlation in over 5,000 patient samples), and that patient survival correlates positively with PKC expression and negatively with PHLPP1 expression in pancreatic cancer. Accordingly, the present disclosure provides that a ratio of PKC protein to PHLPP1 protein in patient tumor samples provides a mechanistic biomarker for the prognostic stratification of pancreatic adenocarincoma patients based on survival rates.

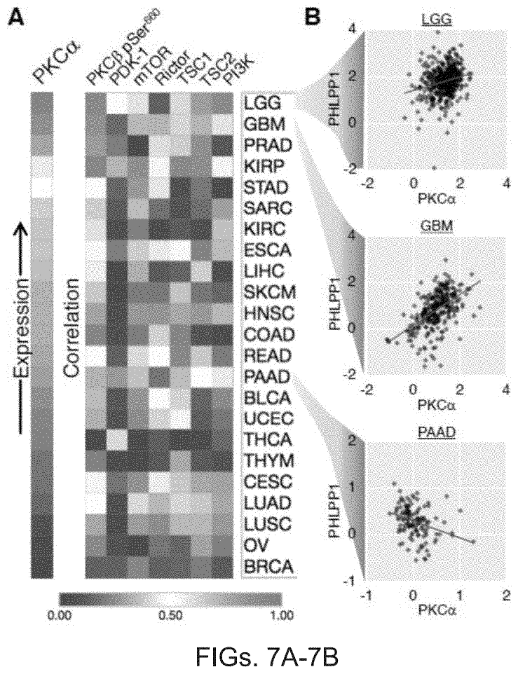

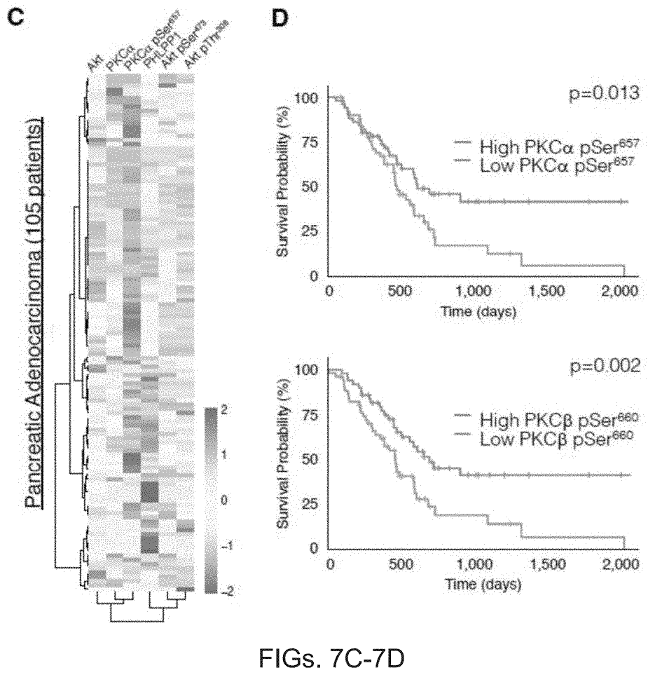

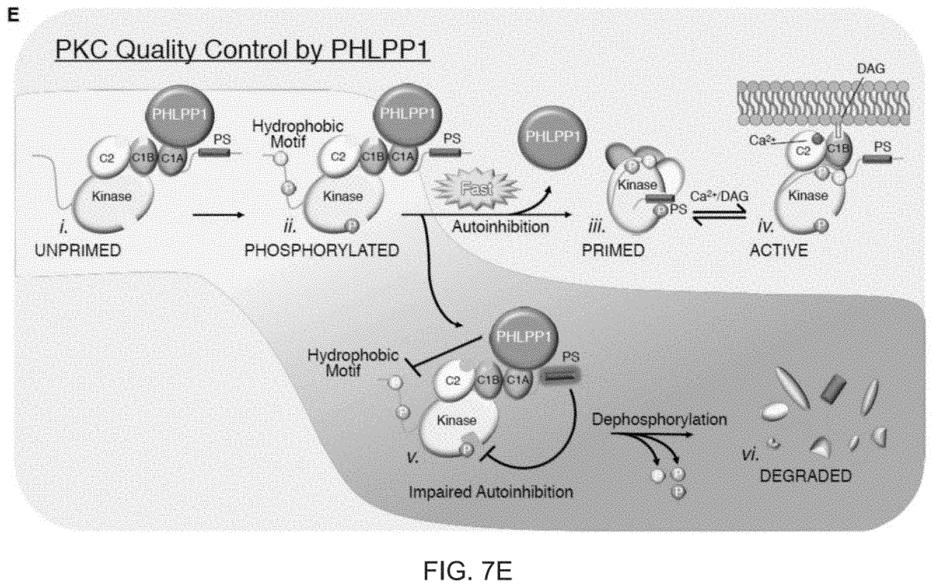

The pseudosubstrate and phosphorylation play an interdependent and essential function in PKC homeostasis that is exploited in cancer to effectively lose PKC. First, phosphorylation of the hydrophobic motif is necessary for the pseudosubstrate-dependent transition of newly-synthesized PKC to the mature, autoinhibited conformation that prevents basal signaling in the absence of second messengers. In this manner, phosphorylation serves as an “off” switch, ensuring that the deregulated activity of newly-synthesized enzyme is immediately quenched (FIG. 7E; iii). This pseudosubstrate-engaged conformation, in turn, is necessary to protect newly-synthesized PKC from dephosphorylation by bound PHLPP1 (FIG. 7E; ii). Because lack of phosphate at the hydrophobic motif results in proteasomal degradation of PKC, PHLPP1 provides a quality control step that prevents aberrant PKC from accumulating in the cell (FIG. 7E; v). The vulnerability of aberrant PKC to dephosphorylation/degradation is exploited in cancer. Notably, the pseudosubstrate is a hotspot for cancer-associated mutations. Those that loosen autoinhibition are LOF because phosphorylation cannot be retained at the hydrophobic motif, resulting in an unstable protein that is degraded (FIG. 7E, vi).

Those that enhance autoinhibition are also LOF by decreasing signaling output, pushing the equilibrium to the closed conformation (FIG. 7, iii). Validating the requirement for phosphorylation at the hydrophobic motif for PKC stability, analysis of over 5,000 tumor samples and 1,500 cell lines reveals an almost 1:1 correlation between total PKC levels and hydrophobic motif phosphorylation. Additionally, we identify PAAD as a malignancy in which PHLPP1 quality control dominates in controlling PKC levels. Consistent with a tumor-suppressive role of PKC, low levels of PKC are associated with poor survival outcome in this cancer. Thus, in PAAD, there is a dependence upon PHLPP1 to suppress PKC expression, providing a potential therapeutic target to stabilize PKC and increase patient survival.

These findings delineate a mechanism that results in the loss of a tumor suppressor at the post-translational level, rather than the prevalent mechanism involving genetic deletion of regions encoding tumor suppressor genes (Weinberg, 1991). Indeed, the tumor-suppressive role of PKC isozymes remained uncharacterized for several decades, in part due to relatively infrequent deletion of PKC genes compared to other notable tumor suppressors. However, impairing protein stability is an equally effective method for LOF, as epitomized by the tumor suppressor p53, which is also stabilized by phosphorylation to prevent its degradation in the context of the DNA damage response (Chehab et al., 1999).

Attesting to its key regulatory role, the hydrophobic motif was recently identified as a hotspot for cancer mutations across most AGC kinases, including PKCβ (Huang et al., 2018). However, phosphate at this PHLPP-regulated position plays distinct roles among these kinases. For Akt and S6K, dephosphorylation at the hydrophobic motif attenuates catalytic activity (Gao et al., 2005; Liu et al., 2011) without affecting stability. This latter point is evident from our own analysis showing no significant correlation between the total levels of these two AGC kinases and phosphorylation of their respective hydrophobic motifs. But in PKC, phosphorylation serves a very different function: it allows the pseudosubstrate to be tethered in the substrate-binding cavity to mediate autoinhibition and promote the stable conformation that results in a long half-life of PKC in cells. Thus, PKC is unique among PHLPP1 hydrophobic motif substrates in that phosphate protects the kinase from degradation.

An unexpected finding from this study is that deletion of the autoinhibitory pseudosubstrate abolished any detectable phosphorylation at the three processing sites yet resulted in constitutively and maximally active PKC. Thus, surprisingly, PKC with no priming phosphates can have full, unrestrained catalytic activity. Furthermore, as is the case for the activation loop phosphate (Sonnenburg et al., 2001), transient phosphorylation at the hydrophobic motif is necessary for PKC to progress to a catalytically-competent conformation, but then becomes dispensable for activity. It was unable to observe even transient phosphorylation of the autoinhibition-deficient PKC during processing in mammalian cells, underscoring the strict homeostatic control of the hydrophobic motif site. Phosphorylation of autoinhibition-deficient PKC, however, was readily observed in Sf9 insect cells. PKC may evade PHLPP quality control in insect cells because of the evolutionary functional divergence of the PHLPP PH domain (Park et al., 2008), a key determinant in its dephosphorylation of PKC in cells (Gao et al., 2008). One possible explanation for the requirement of negative charge at the hydrophobic motif early in the life cycle of PKC is that autophosphorylation of this site triggers association of the C-tail with the kinase domain, an important step in aligning the regulatory spine (Taylor and Komev, 2011).

Although protein kinases share a common active conformation, numerous mechanisms of autoinhibition have evolved to maintain kinases in inactive states (Bayliss et al., 2015). For nearly all protein kinases, phosphorylation serves to relieve autoinhibition, usually elicited by binding to regulatory molecules following agonist stimulation. For example, Akt autoinhibition by the PH domain is relieved via binding phosphatidylinositol-3,4,5-trisphosphate (PIP3) to promote activating phosphorylations at the activation loop and hydrophobic motif (Alessi et al., 1996).

Indeed, oncogenic mutations that dislodge the PH domain from the kinase domain activate Akt independently of PIP3 generation (Parikh et al., 2012). In a similar manner, we find that cancer-associated PKC mutations in the pseudosubstrate also elicit constitutive activity. However, by impairing autoinhibition, these mutations induce PHLPP1-dependent dephosphorylation and are effectively LOF by promoting PKC degradation. Thus, cancer-associated activating mutations that disrupt autoinhibition present as gain-of-function mutations in Akt but manifest as LOF mutations in PKC.

The role of PHLPP1 quality control in setting the level of PKC in cells has important ramifications for cancer therapies, as higher expression levels of PKC isozymes have been reported to predict improved patient survival in diverse malignancies (Newton, 2018). For example, higher levels of PKCa and PKCIIl protein predict improved outcome in T-cell acute lymphoblastic leukemia (T-ALL) and colorectal cancer, respectively (Dowling et al., 2016; Milani et al., 2014). Here, we show that high PKC hydrophobic motif phosphorylation correlated with dramatically increased survival in PAAD. Because greater than 90% of pancreatic cancers harbor an activating K-Ras mutation (Almoguera et al., 1988), one possibility is that high PKC levels suppress K-Ras signaling. Consistent with this, PKC phosphorylation of K-Ras on Ser¹⁸¹ in the famesyl-electrostatic switch was reported to disengage K-Ras from the plasma membrane (Bivona et al., 2006).

Although the role of this specific PKC phosphorylation in tumors is unclear (Barcelo et al., 2014), McCormick and colleagues have shown that oral administration of a phorbol ester with very weak potency promoted K-Ras phosphorylation and repressed growth in orthotopic mouse models of human pancreatic cancer (Wang et al., 2015). Furthermore, PKC suppresses growth of oncogenic K-Ras driven tumors in a xenograft mouse model of colorectal adenocarcinoma, and deletion or mutation of only one PKC allele is sufficient to enhance tumor growth (Antal et al., 2015b). Additionally, K-Ras is among the most frequently co-mutated genes in tumors with LOF PKC mutations (Antal et al., 2015b). Together, these data support a role for functional PKC in suppressing oncogenic K-Ras signaling. Another mechanism by which PKC suppresses oncogenic signaling was recently unveiled by Black and coworkers, who showed that PKCa deficiency in endometrial tumors enhances oncogenic Akt signaling via a mechanism involving its modulation of the activity of a PP2A family phosphatase (Hsu et al., 2018). Thus, targeting the PHLPP1-dependent quality control step of PKC processing may be a promising approach to stabilize PKC in cancers involving oncogenes controlled by PKC.

The present disclosure underscores the importance of careful consideration of PKC phosphorylation mechanisms in cancer therapies. Notably, mTOR kinase inhibitors and Hsp90 inhibitors currently in clinical trials will have the unwanted result of preventing PKC processing, thus depleting levels of this tumor suppressor. Coupling such therapies with disruption of PHLPP1-dependent quality control may have significant therapeutic benefit. Thus, the post-translational inactivation of PKC by PHLPP1, distinct from loss of other tumor suppressors via genetic mechanisms, presents a druggable interaction and potential vulnerability in cancers that respond to PKC restoration.

### Methods for Diagnosing and/or Prognosing Cancer in A SUBJECT

In one aspect, disclosed herein is a method for diagnosing or prognosing a cancer in a subject, the method including at least the steps of (a) obtaining a sample from the subject, (b) measuring a level of at least one biomarker in the subject, and (c) comparing the level of the at least one biomarker in the subject to a level of the at least one biomarker in a control sample, wherein the control sample is from a subject who does not have cancer and wherein the at least one biomarker is selected from PKC, PHLPP1, or the ratio of PKC/PHLPP1 in the sample.

In an aspect, when the biomarker is PKC, the level of the biomarker can be higher than the level of the same biomarker in a control sample; in a further aspect, a higher PKC level in a patient is associated with an increased cancer survival rate.

In an alternative aspect, when the biomarker is PKC, the level of the biomarker can be lower than the level of the same biomarker in a control sample; in a further aspect, a lower PKC level in a patient is associated with a reduced cancer survival rate.

In another aspect, when the biomarker is PHLPP1, the level of the biomarker can be lower than the level of the same biomarker in a control sample; in a further aspect, a lower PHLPP1 level in a patient is associated with an increased cancer survival rate. In an alternative aspect, when the biomarker is PHLPP1, the level of the biomarker can be higher than the level of the same biomarker in a control sample; in a further aspect, a higher PHLPP1 level in a patient is associated with a reduced cancer survival rate.

In still another aspect, when the biomarker is the ratio of PKC to PHLPP1, the ratio can be higher than the ratio in a control sample; in a further aspect, a higher PKC/PHLPP1 ratio in a patient is associated with an increased cancer survival rate.

In an alternative aspect, when the biomarker is the ratio of PKC to PHLPP1, the ratio can be lower than the ratio in a control sample; in a further aspect, a lower PKC/PHLPP1 ratio in a patient is associated with a reduced cancer survival rate. In a further aspect, the ratio of PKC to PHLPP can be higher than about 1:1 (i.e., associated with increased cancer survival), or can be about 1:1, or can be lower than about 1:1 (i.e., associated with decreased cancer survival).

In any of these aspects, if biomarker levels in a subject indicate a reduced cancer survival rate, medical personnel may recommend a more aggressive course of treatment. In another aspect, if biomarker levels in a subject indicate an increased cancer survival rate, milder treatments with fewer systemic side effects may suffice for treating the subject.

In one aspect, the cancer can be selected from pancreatic adenocarcinoma, colon cancer, breast cancer, ovarian cancer, Wilms tumor, prostate cancer, hepatocellular carcinoma, glioblastoma multiforme, kidney renal papillary cell carcinoma, chronic myelogenous leukemia, non-small cell lung cancer, diffuse large B-cell lymphoma, chronic lymphocytic leukemia, renal cell carcinoma, bladder cancer, melanoma, low grade glioma, or any combination thereof. In one aspect, the cancer is pancreatic adenocarcinoma or another pancreatic cancer.

In any of these aspects, the sample can be whole blood, serum, plasma, a fine needle biopsy sample from a tumor, a fine needle aspirate sample from a tumor, a core needle biopsy sample from a tumor, an excisional biopsy sample, or any combination thereof.

### Methods for Measuring Biomarkers

In one aspect, when the biomarker is PKC, the level of the at least one biomarker can be measured by (a) contacting the sample with an anti-PKC antibody, (b) determining an amount of antibody binding using an antibody quantification technique, and (c) correlating the amount of antibody binding to the level of PKC in the sample. In another aspect, when the biomarker is PHLPP1, the level of the at least one biomarker can be measured by (a) contacting the sample with an anti-PHLPP1 antibody, (b) determining an amount of antibody binding using an antibody quantification technique, and (c) correlating the amount of antibody binding to the level of PHLPP1 in the sample.

In still another aspect, when the biomarker is the ratio of PKC to PHLPP1, the level of the at least one biomarker can be measured by (a) contacting the sample with an anti-PKC antibody, (b) contacting the sample with an anti-PHLPP1 antibody, (c) determining the amount of anti-PKC antibody binding and anti-PHLPP1 antibody binding using an antibody quantification technique, (d) correlating the amount of antibody binding to the levels of PCK and PHLPP1 in the sample, respectively, and E calculating a ratio of PCK to PHLPP1.

In any of these aspects, the antibody quantification technique can be selected from immunofluorescence, radiolabeling, immunoblotting, Western blotting, enzyme-linked immunosorbent assay, flow cytometry, immunoprecipitation, immunohistochemistry, biofilm test, affinity ring test, antibody array optical density test, chemiluminescence, or any combination thereof.

### Method for Treating Cancer

In one aspect, disclosed herein is a method for treating cancer in a subject, the method including at least the steps of (a) obtaining a sample from the subject, (b) measuring a level of at least one biomarker in the subject, (c) comparing the level of the at least one biomarker in the subject to a level of the at least one biomarker in a control sample, and (d) administering an effective amount of a PHLPP1 inhibitor to the subject, wherein the control sample is from a subject who does not have cancer, and wherein the at least one biomarker is selected from PKC, PHLPP1, or a ratio of PCK to PHLPP1.

In one aspect, the PHLPP1 inhibitor can be NSC117079, NSC45586, or any combination thereof. In another aspect, the cancer can be pancreatic adenocarcinoma or another pancreatic cancer.

### EXAMPLES

### EXAMPLE 1

### Experimental Model and Subject Details

### Cell Culture and Transfection

COS7 cells, Phlpp1+/+MEFs, and Phlpp1-/−MEFs were cultured in DMEM (Coming) containing 10% fetal bovine serum (Atlanta Biologicals) and 1% penicillin/streptomycin (GIBCO) at 37° C. in 5% CO2. Generation of the PHLPP1 MEFs was described previously (Masubuchi et al., 2010). Transient transfection was carried out using the Lipofectamine 3000 Transfection Reagent (Thermo Fisher Scientific). Sf9 cells were grown in Sf-900 II SFM media (GIBCO) in shaking cultures at 27° C.

### Plasmids and Constructs

The C Kinase Activity Reporter (CKAR) was previously described (Violin et al., 2003). PKC pseudosubstrate-deleted constructs were generated by looping out the 54 bases comprising residues 19-36 of PKCa or PKCβII by QuikChange Mutagenesis (Agilent). Scrambled and Neutral Pseudosubstrate constructs were generated by QuikChange Mutagenesis (Agilent). The catalytic domain was generated by cloning residues 296-673 of human PKCβII into pcDNA3 containing an N-terminal HA tag at the Notl and Xbal sites.

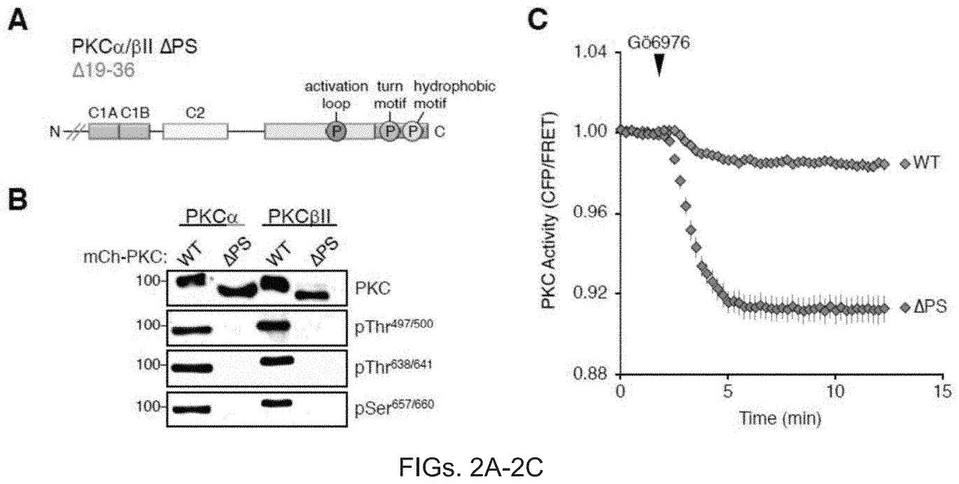

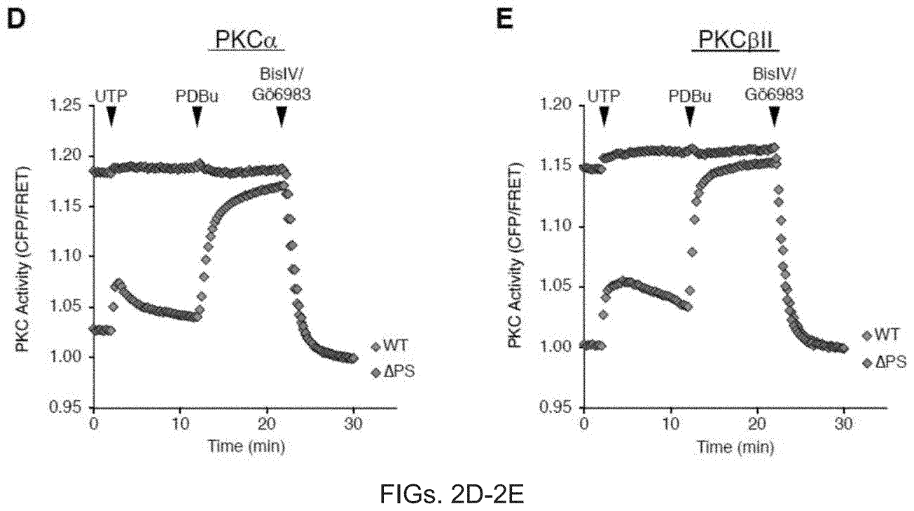

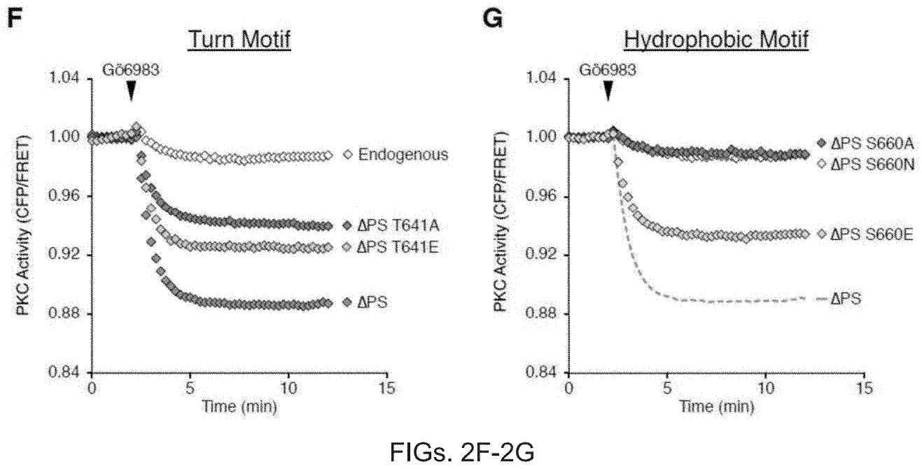

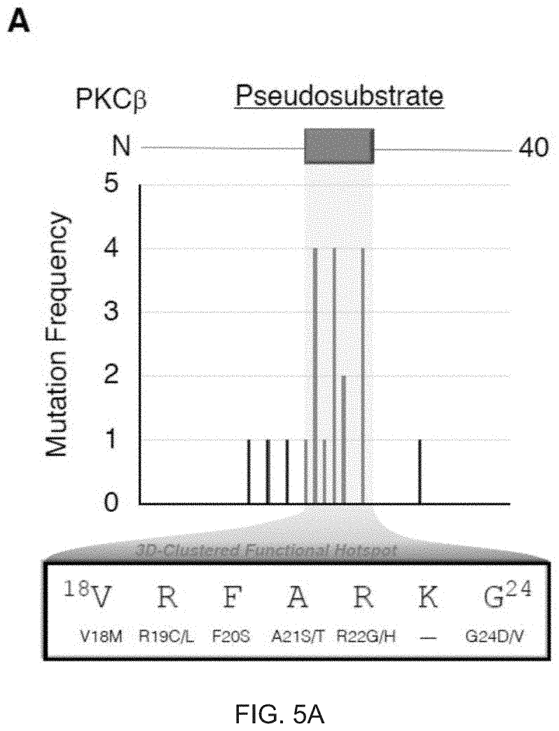

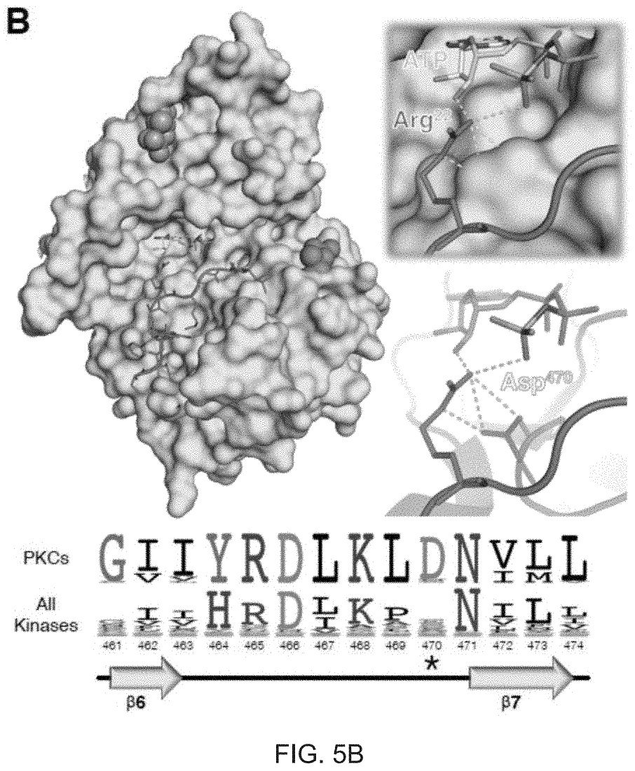

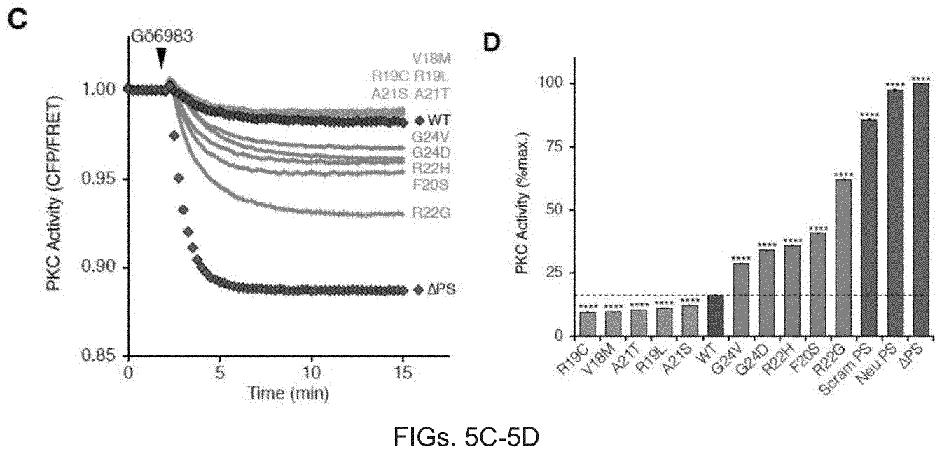

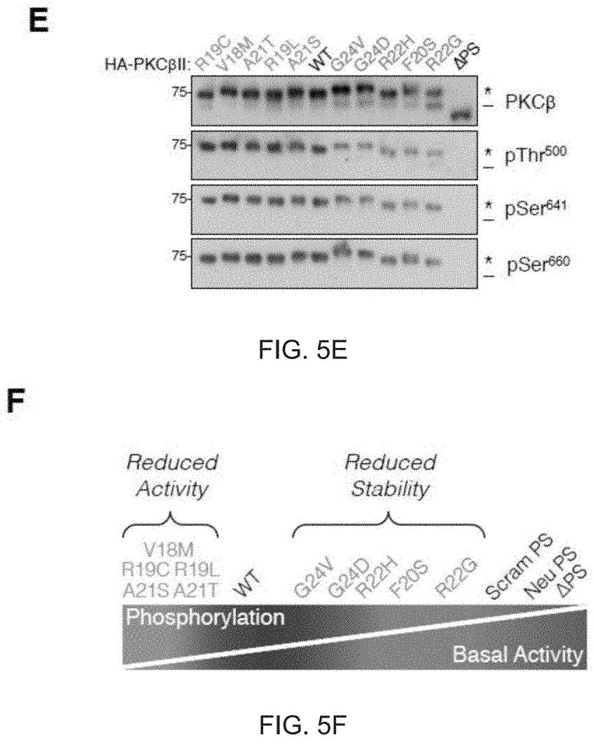

Regulatory domain constructs were generated by cloning residues 1-295 of human PKCβ into pcDNA3 with mCherry at the N terminus at the BamHl and Xbal sites. mCherry-tagged constructs were cloned into pcDNA3 with mCherry at the N terminus at the BamHl and Xbal sites. mYFP-tagged constructs were cloned into pcDNA3 with mYFP at the N terminus at the Xhol and Xbal sites. HA-tagged rat PKCβII constructs were cloned into pcDNA3 with HA at the N terminus at the Notl and Xbal sites. HA-tagged human PKCβII constructs were cloned into pcDNA3 with HA at the N terminus at the Xhol and Xbal sites. Kinameleon was cloned into pcDNA3 as mYFP-PKCβII-mCFP. All mutants were generated by QuikChange Mutagenesis (Agilent). Rat PKC constructs were used with the exception of human PKCa in FIG. 2D and human PKCpil in FIGS. 5C, 5D, and 5E.

### FRET Imaging and Analysis

Cells were imaged as described previously (Gallegos et al., 2006). For activity experiments COS7 cells were co-transfected with the indicated mCherry-tagged PKC construct and CKAR. For Kinameleon experiments, the indicated Kinameleon construct containing mYFP and mCFP was transfected alone. For translocation experiments, COS7 cells were co-transfected with the indicated mYFPtagged construct and plasma-membrane targeted mCFP at a ratio of 10:1. Baseline images were acquired every 15 s for 2 min prior to ligand addition. F6rster resonance energy transfer (FRET) ratios represent mean±SEM from at least three independent experiments.

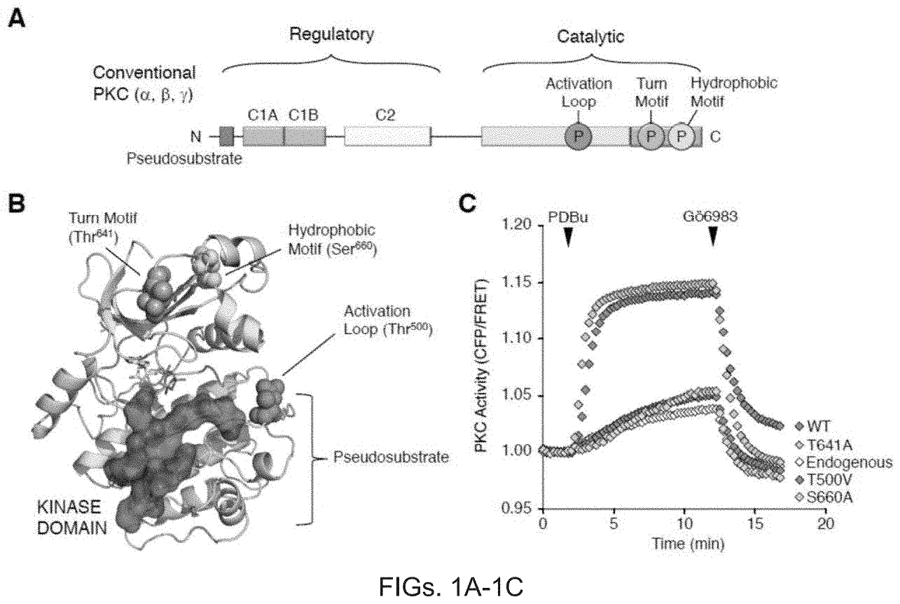

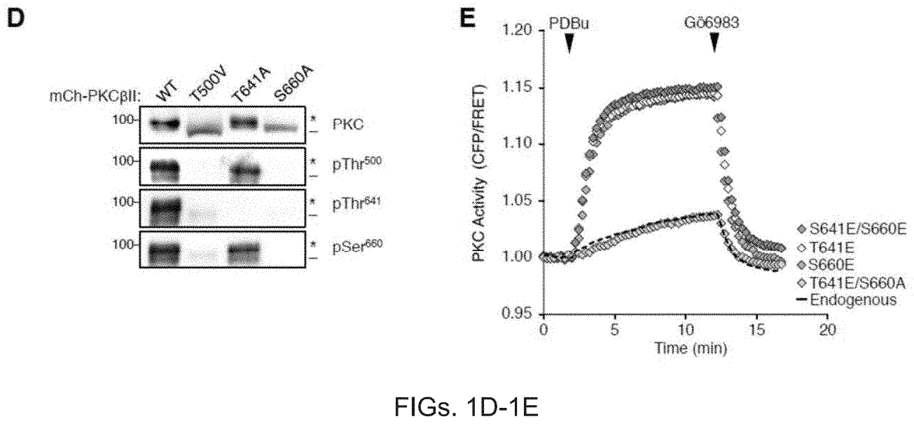

All data were normalized to the baseline FRET ratio of each individual cell unless noted that absolute FRET ratio was plotted or traces were normalized to levels post-inhibitor addition. When comparing translocation kinetics, data were also normalized to the maximal amplitude of translocation for each, as previously described, in order to compare translocation rates (Antal et al., 2014). Every experiment contained an mCherry-transfected control to measure endogenous activity, and an mCherry-tagged WT (or deleted pseudosubstrate) PKC. Control traces are depicted as dotted lines: specifically, the endogenous trace in FIG. 1E and the deleted pseudosubstrate trace in FIGS. 2G and 31, were redrawn to serve as a point of reference in those figures (data were acquired and derived in the same experiments which generated all the data in FIGS. 1A-1E and FIGS. 2A-2G, respectively).

### Immunoblotting and Antibodies

Cells were lysed in PPHB: 50 mM NaPO4 (pH 7.5), 1% Triton X-100, 20 mM NaF, 1 mM Na4P207, 100 mM NaCl, 2 mM EDTA, 2 mM EGTA, 1 mM Na3VO4, 1 mM PMSF, 40 mg/mL leupeptin, 1 mM DTT, and 1 mM microcystin. Phlpp1+/+ and Ph/pp1-/−MEFs were lysed in 50 mM Tris (pH 7.4), 1% Triton X-100, 50 mM NaF, 10 mM Na4P207, 100 mM NaCl, 5 mM EDTA, 1 mM Na3VO4, 1 mM PMSF, 40 mg/mL leupeptin, and 1 mM microcystin. Triton-soluble fractions were analyzed by SDS-PAGE on 7% big gels to observe phosphorylation shift, transfer to PVDF membrane (Biorad), and western blotting via chemiluminescence SuperSignal West reagent (Thermo Fisher) on a FluorChem Q imaging system (ProteinSimple). In western blots, the asterisk (*) denotes the position of mature, phosphorylated PKC; whereas, the dash (−) indicates the position of unphosphorylated PKC. The turn motif and hydrophobic motif phosphorylations, but not the activation loop phosphorylation, induces an electrophoretic mobility shift that retards the migration of the phosphorylated species. The pan anti-phospho-PKC activation loop antibody (PKC pThroo) was described previously (Dutil et al., 1998). The anti-phospho-PKCa/pil turn motif (pT638/641; 9375S) and pan anti-phospho-PKC hydrophobic motif (PIl pS° ”; 9371S) antibodies and Calyculin A were purchased from Cell Signaling. Anti-PKCβ (610128) and PKCa (610128) antibodies were purchased from BD Transduction Laboratories. The DsRed antibody was purchased from Clontech. The anti-PHLPP1 antibody was purchased from Proteintech (22789-1-AP). The anti-HA antibody for immunoblot was purchased from Roche. The anti-HA (clone 16B12; 901515) and anti-FLAG (Clone L5; 637301) antibodies used for immunoprecipitation were purchased from BioLegend. The anti-a-tubulin (T6074) and anti-His (H1029) antibodies were from Sigma.

### Baculovirus Expression of PKC and PHLPP1 PP2C

Human PKCβII, PKCβII DPS, and PHLPP1 PP2C (residues 1154-1422) were cloned into the pFastBac vector (Invitrogen) containing an N-terminal GST or His tag. Using the Bac-to-Bac Baculovirus Expression System (Invitrogen), the pFastBac plasmids were transformed into DH10Bac cells, and the resulting bacmid DNA was transfected into Sf9 insect cells via CelIFECTIN (ThermoFisher Scientific). Sf9 cells were grown in Sf-900 II SFM media (GIBCO) in shaking cultures at 27° C. The recombinant baculoviruses were harvested and amplified. Sf9 cells were seeded in 35 mm dishes (1×106 cells/dish) and infected with baculovirus. Following 2 days of incubation, Sf9 cells were lysed directly in 1 x Laemmli sample buffer, sonicated, and boiled at 95° C. for 5 min.

### Pulse-Chase Experiments

For pulse-chase experiments, COS7 cells were incubated with Met/Cys-deficient DMEM for 30 min at 37° C. The cells were then pulse-labeled with 0.5 mCi/mL [³⁵S]Met/Cys in Met/Cys-deficient DMEM for 7 min at 37° C., media were removed, washed with dPBS (Coming), and chased with DMEM culture media (Coming) containing 200 mM unlabeled methionine and 200 mM unlabeled cysteine. At the indicated times, cells were lysed in PPHB and centrifuged at 13,000×g for 3 min at 22° C., supematants were pre-cleared for 30 min at 4° C. with Protein A/G Beads (Santa Cruz), and protein complexes were immunoprecipitated from the supernatant with either an anti-HA or anti-FLAG monoclonal antibody (BioLegend, 16B12; BioLegend L5) overnight at 4° C. The immune complexes were collected with Protein A/G Beads (Santa Cruz) for 2 hr, washed 3x with PPHB, separated by SDS-PAGE, transferred to PVDF membrane (Biorad), and analyzed by autoradiography and Western blot. Co-immunoprecipitation experiments were performed similarly, omitting the labeling and autoradiography steps.

### Reverse Phase Protein Array

For RPPA experiments, patient samples and cell line samples were prepared and antibodies were validated as described previously (Li et al., 2017; Tibes et al., 2006).

### Cancer Mutation Identification

Cancer-associated pseudosubstrate mutations were identified by querying the cBioPortal for Cancer Genomics, the Catalogue of Somatic Mutations in Cancer (COSMIC), mutation3D at the Cornell University Weill Institute for Cell and Molecular Biology, and the International Cancer Genome Consortium (ICGC) databases.

### QUANTIFICATION AND STATISTICAL ANALYSIS

Statistical significance was determined via Repeated-measures One-Way ANOVA and Brown-Forsythe Test or Student's t test performed in GraphPad Prism 6.0 a (GraphPad Software). The half-time of translocation or degradation was calculated by fitting the data to a non-linear regression using a one-phase exponential association equation with GraphPad Prism 6.0 a (GraphPad Software).

Western blots were quantified by densitometry using the AlphaView software (Protein Simple).

### Example 2

PKC Priming Phosphorylations Are Necessary for Maturation and Activity PKC priming phosphorylations (FIGS. 1A and 1B) have been presumed to be necessary for catalytic competence based on biochemical studies (Bomancin and Parker, 1997; Cazaubon et al., 1994; Edwards and Newton, 1997; Orr and Newton, 1994). To assess whether phosphorylation at these sites is also necessary in a cellular context, we measured the agonist-evoked activity of wild-type (WT) PKCβII or mutants with non-phosphorylatable residues at each of the three priming sites in cells using the C kinase activity reporter (CKAR) (Violin et al., 2003). Phorbol 12,13-dibutyrate (PDBu) treatment caused a robust increase in CKAR phosphorylation in COS7 cells expressing WT PKC or turn motif mutant (T641A) that was reversed by addition of PKC inhibitor (FIG. 1C).

In contrast, cells expressing activation loop (T500V) or hydrophobic motif (S660A) mutants displayed no increase in CKAR phosphorylation above that of endogenous PKC. Thus, phosphorylatable residues at the activation loop and hydrophobic motif, but not turn motif, are necessary for cellular PKC activity. Western blot analysis with phospho-specific antibodies revealed that WT PKCβII protein was phosphorylated at the C-terminal sites (causing an electrophoretic mobility shift [asterisk]; Keranen et al., 1995) and at the activation loop (FIG. 1D).

The T641A protein was phosphorylated at the activation loop and hydrophobic motif, whereas the T500V and S660A proteins were unphosphorylated at all three sites, exhibited by their faster mobility (dash) and lack of reactivity with phosphospecific antibodies (FIG. 1D).

To assess whether negative charge at the hydrophobic motif is sufficient for cellular PKC activity, the PDBu-stimulated activity of phosphomimetic PKC mutants with Glu substitutions at either or both of the C-tail phosphorylation sites was examined (FIG. 1E). Replacement with Glu at the turn motif (T641E), hydrophobic motif (S660E), or both C-terminal sites (T641E/S660E) resulted in activation kinetics comparable to those observed with WT PKCβII (see FIG. 1C).

In contrast, PKCβII T641E/S660A was inactive, revealing a requirement for negative charge at the hydrophobic motif irrespective of turn motif phosphorylation.

Thus, phosphorylation of the activation loop and hydrophobic motif, but not the turn motif, is necessary for PKC maturation and enzymatic activity in cells.

### Example 3

### The Autoinhibitory Pseudosubstrate Is Required for Cellular PKC Phosphorylation

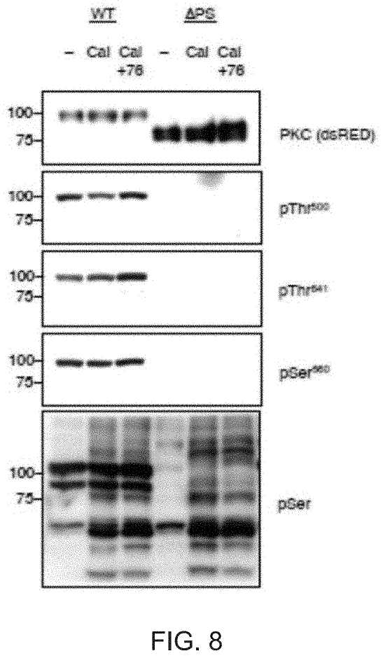

Extensive in vitro biochemical studies have established that the pseudosubstrate is necessary to restrain PKC activity in the absence of second messengers (House and Kemp, 1987; Orr et al., 1992; Pears et al., 1990). To probe the role of the pseudosubstrate in a cellular context, the 18-amino-acid-pseudosubstrate segments of two cPKC isozymes, PKCa and PKCβII were deleted (FIG. 2A; PKCaDPS and PKCβII DPS) and the phosphorylation state and cellular activity of the expressed proteins were examined. Deletion of the pseudosubstrate abolished phosphorylation at all three priming sites (FIG. 2B), which could not be rescued by treatment with the phosphatase inhibitor Calyculin A (FIG. 8). Surprisingly, however, analysis of PKC basal activity, assessed by the drop in CKAR phosphorylation upon addition of PKC inhibitor, revealed that PKCβII DPS had high basal activity despite the absence of priming phosphorylations (FIG. 2C). Whereas both WT PKCa and PKCβII were activated by treatment of cells with uridine triphosphate (UTP) and PDBu, neither PKCaDPS nor PKCβII DPS responded to either agonist, but constitutive, maximal activity was revealed upon inhibitor addition (FIGS. 2D and 2E). Thus, deletion of the pseudosubstrate results in constitutively active PKC that, unexpectedly, has maximal activity in the absence of priming phosphorylations.

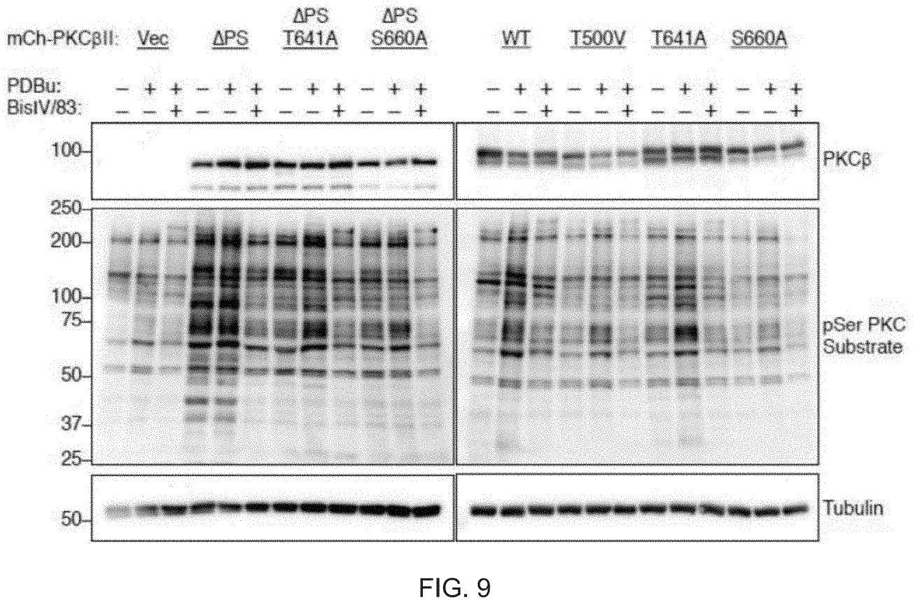

Given that replacement of the hydrophobic motif Ser with Ala abolished cellular PKC activity (FIG. 1C), phosphorylation mutants of PKCβII DPS (which is not phosphorylated and constitutively active) were used to assess whether negative charge at the hydrophobic motif is required in the maturation of PKC but becomes dispensable thereafter. Turn motif mutants PKCβII DPS T641A and PKCβII DPS T641E had enhanced basal activity with little preference for Ala versus Glu (FIG. 2F). In contrast, only the phosphomimetic Glu, but not Ala or Asn, at the hydrophobic motif site conferred activity (FIG. 2G). Mutation of the hydrophobic motif did not simply alter substrate specificity to abolish recognition of CKAR; western blot analysis using a phospho-Ser PKC substrate antibody revealed no significant phosphorylation above basal levels in cells expressing PKCβII DPS S660A but robust phosphorylation in cells expressing PKCβII DPS (FIG. 9). These data reveal that deletion of the pseudosubstrate (1) results in a constitutively active PKC that retains no phosphorylation at the priming sites and (2) does not bypass the requirement for negative charge at the hydrophobic motif to gain catalytic competence.

### Example 4

### The Autoinhibited Conformation of PKC Retains Priming Phosphorylations

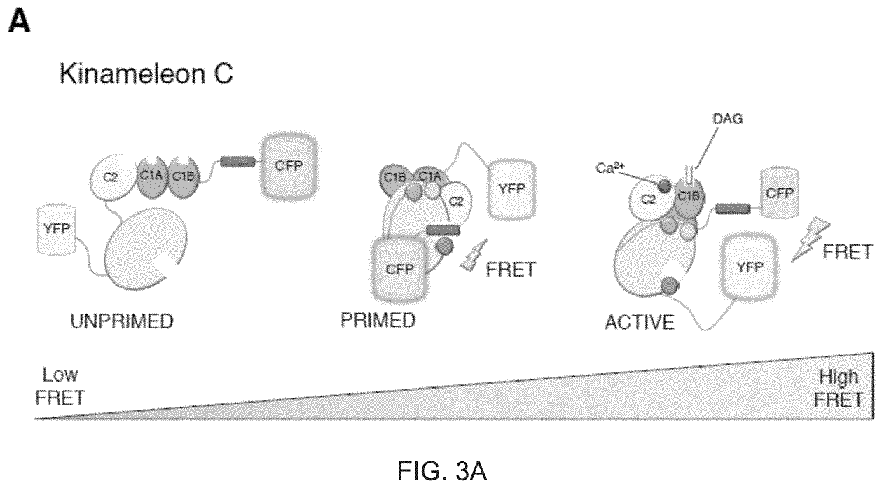

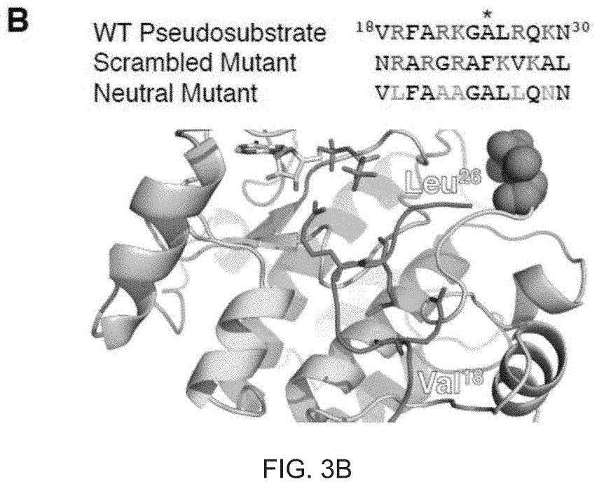

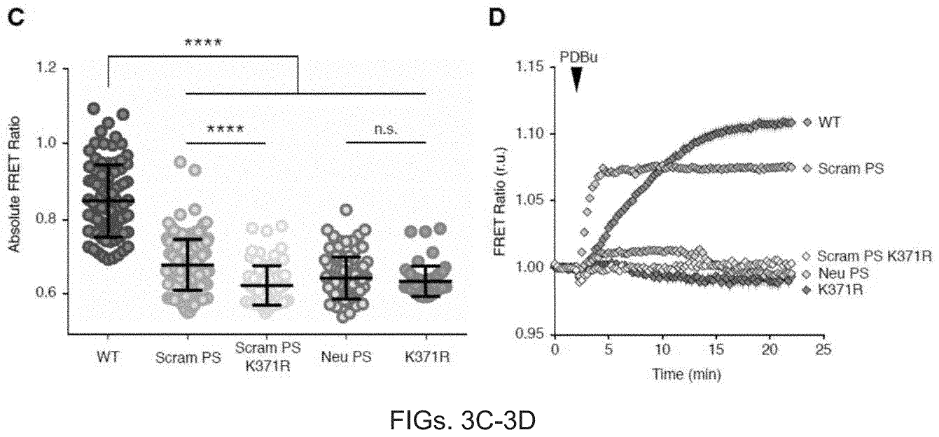

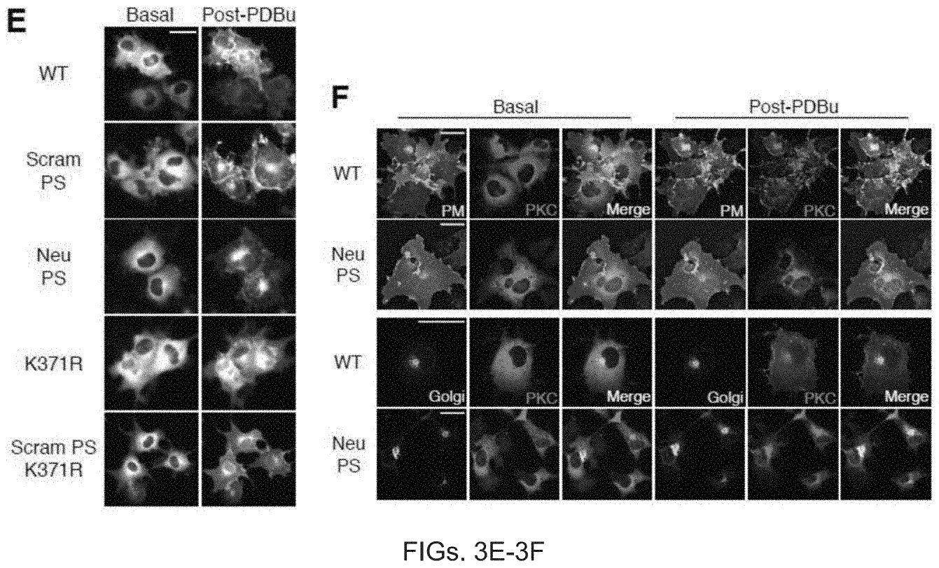

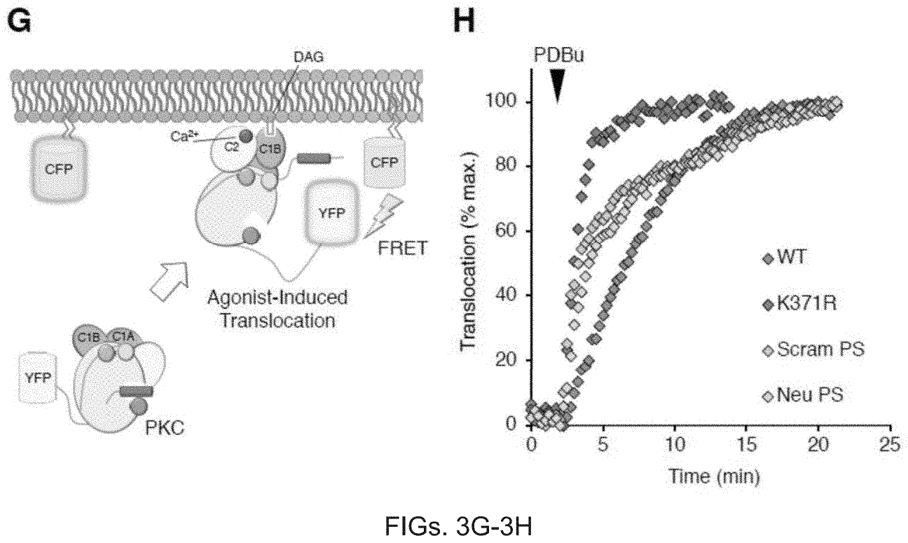

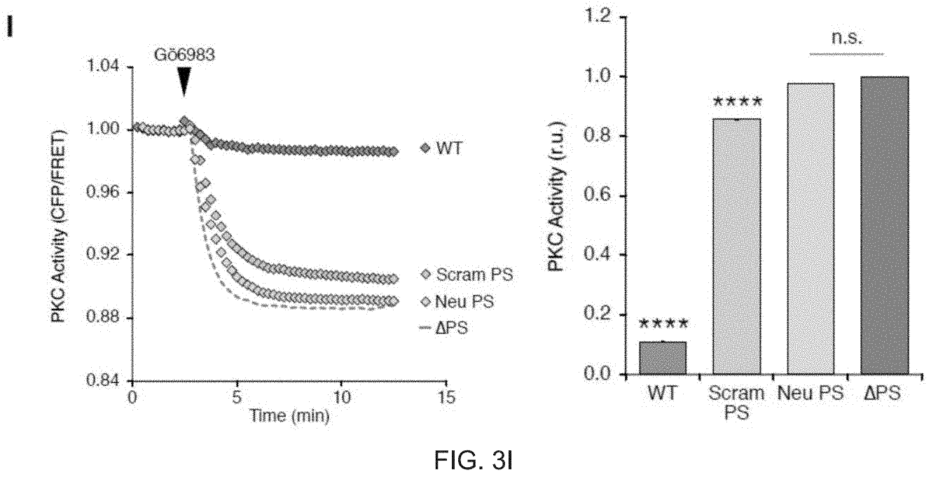

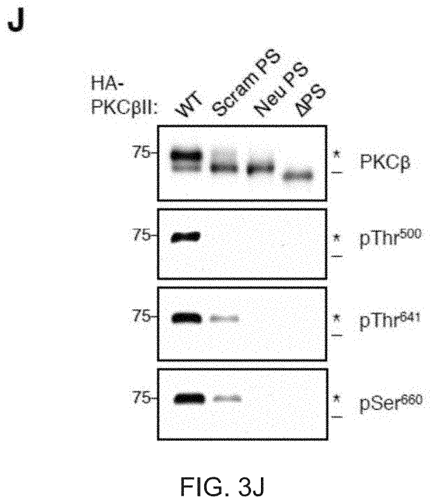

To explore the relationship between PKC phosphorylation and activity, a PKC conformation reporter, Kinameleon, was used, wherein intramolecular rearrangements within PKC alter Fbrster resonance energy transfer (FRET) between flanking cyan fluorescent protein (CFP) and yellow fluorescent protein (YFP) molecules (FIG. 3A). Using this reporter, it was showed previously that PKC adopts at least three distinct conformations: (1) a low-FRET unprimed state, in which the regulatory domains are exposed; (2) an intermediate-FRET primed state, in which PKC is fully phosphorylated at the C-terminal sites and autoinhibited, with the regulatory domains masked; and (3) a high-FRET active state, in which the regulatory domains are engaged on the plasma membrane (FIG. 3A; Antal et al., 2014). To examine how autoinhibition affects PKC conformation, the affinity of the pseudosubstrate for the kinase domain was altered by either scrambling the position of positively charged amino acids in the pseudosubstrate (FIG. 3B; scrambled mutant) or replacing them with neutral residues (FIG. 3B; neutral mutant) in the Kinameleon reporter (see SEQ ID NOs: 1-3).

Analysis of basal FRET, indicative of the average conformation of the PKC embedded in the reporter, revealed that the scrambled (PKCβII Scram PS) and neutral (PKCβII Neu PS) pseudosubstrate mutants had significantly lower basal FRET ratios than WT PKCβII, consistent with an unprimed, open conformation (FIG. 3C). However, the scrambled mutant displayed modest propensity to autoinhibit; introduction of a kinase-dead mutation in PKCβII Scram PS (Scram PS K371R) which abolishes hydrophobic motif autophosphorylation and induces the unprimed conformation) (Antal et al., 2014; Behn-Krappa and Newton, 1999), further reduced the FRET ratio (FIG. 3C). The FRET ratio of PKCβII Neu PS was indistinguishable from that of kinase-dead PKC (K371R). Next, the ability of these mutants to adopt the active conformation upon agonist stimulation was examined.

PDBu treatment caused an increase in FRET ratio for WT PKCβII, reflecting the conformational rearrangement of the N and C termini (FIG. 3D). PKCβII Scram PS underwent a more rapid conformational transition, and the FRET change plateaued at a lower amplitude (FIG. 3D). No conformational change was observed for kinase-inactive PKCβII Scram PS K371R, PKCβII Neu PS, or PKCβII K371R (FIG. 3D).

These data suggest that while PKCβII Scram PS is loosely autoinhibited and rapidly adopts the open/active conformation in the presence of agonist, PKCβII Neu PS resembles unprimed PKC and is incapable of transitioning to the active state. Upon agonist stimulation, cPKC isozymes translocate to the plasma membrane where their C2 domain recognizes PIP2 and C1B domain binds DAG. However, PKC that has not been properly processed by phosphorylation exists in an open conformation with unmasked C1 domains, resulting in localization to DAG-rich Golgi membranes (Antal et al., 2014; Scott et al., 2013). PKCpil Kinameleon reporter proteins that were incapable of acquiring or retaining priming phosphorylations (Neu PS, K371R, Scram PS K371R) translocated primarily to intracellular compartments resembling Golgi membranes following PDBu stimulation (FIG. 3E). In contrast, PKCpil that was fully (WT) or partially (Scram PS) phosphorylated/autoinhibited primarily distributed to the plasma membrane (FIG. 3E). Co-localization analysis between PKC and membrane-targeted CFP confirmed that PKC@ll Neu PS co-distributed with the Golgi marker and WT PKC@ll co-distributed with the plasma membrane marker upon PDBu stimulation (FIG. 3F).

Thus, disruption of the pseudosubstrate unmasks the C1 domains to promote interaction with Golgi membranes. To assess exposure of the C1 domains in the pseudosubstrate mutants, FRET was used to monitor real-time translocation of YFP-tagged PKC to plasma membrane-targeted CFP in live cells (FIG. 3G). In response to PDBu, PKCpil Scram PS and PKC@ll Neu PS translocated to the plasma membrane with significantly faster kinetics than WT PKCpil (FIG. 3H; t1/2 =2.3±0.1 min and 3.1±0.2 min, respectively, versus 5.0 t 0.2 min), but with slower kinetics than kinase-dead PKCpil K371R (FIG. 3H; t1/2=1.0 t 0.1 min), which has fully exposed C1 domains. The accelerated membrane translocation and enhanced affinity for Golgi membranes of the pseudosubstrate mutants support an unprimed, open conformation.

Next, the relationship between pseudosubstrate-dependent conformational changes and PKC activity was assessed using CKAR (FIG. 3I). Addition of PKC inhibitor caused a minimal decrease in CKAR phosphorylation in cells expressing WT PKCpil, indicating low basal activity and effective autoinhibition, but a large decrease in cells expressing PKC@ll Neu PS or PKCpil DPS, reflecting high basal activity and no autoinhibition. PKCpil Scram PS had slightly lower basal activity than that of the constitutively active PKC@ll Neu PS, consistent with weak autoinhibition.

Lastly, whereas WT PKCβII was phosphorylated at all three priming sites, the pseudosubstrate mutants had impaired phosphorylation; the weakly autoinhibited PKCβII Scram PS was minimally phosphorylated, while the unprimed PKCβII Neu PS and PKCβII DPS had no detectable phosphorylation (FIG. 3J). Thus, the degree of PKC phosphorylation correlates with the extent of autoinhibition.

### Example 5

### Autoinhibition Protects PKC from PHLPP1-Mediated Dephosphorylation and Degradation

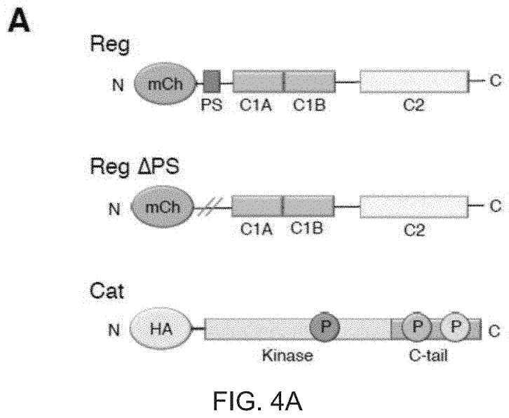

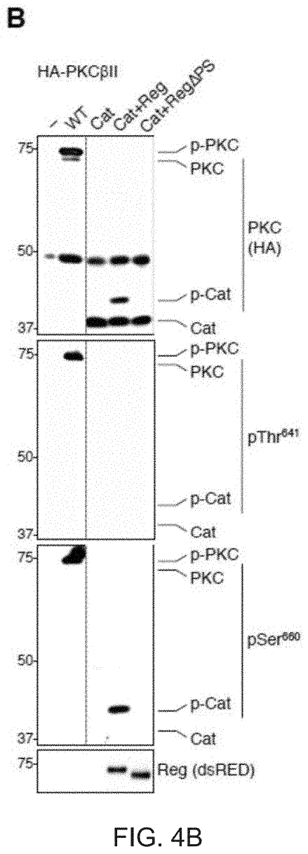

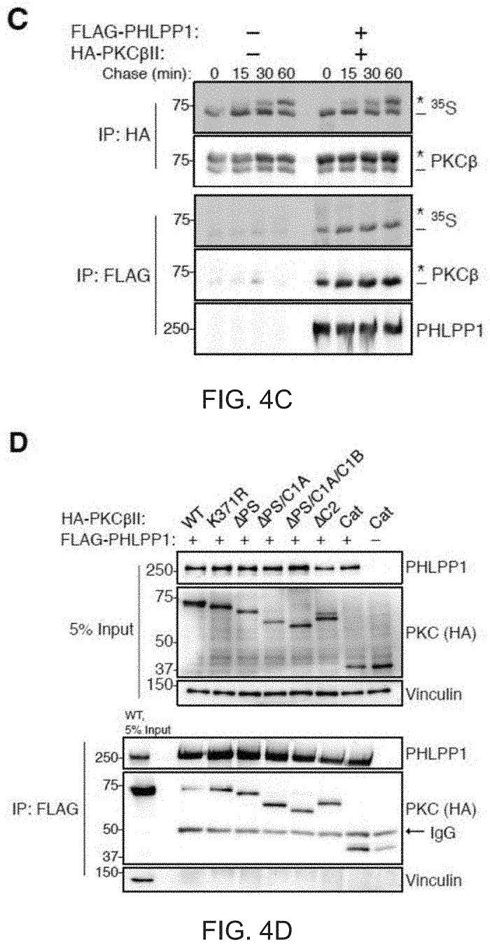

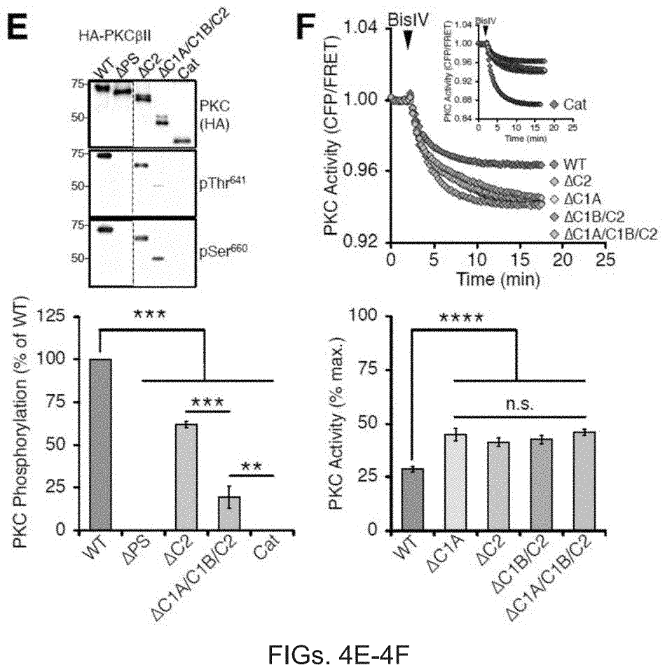

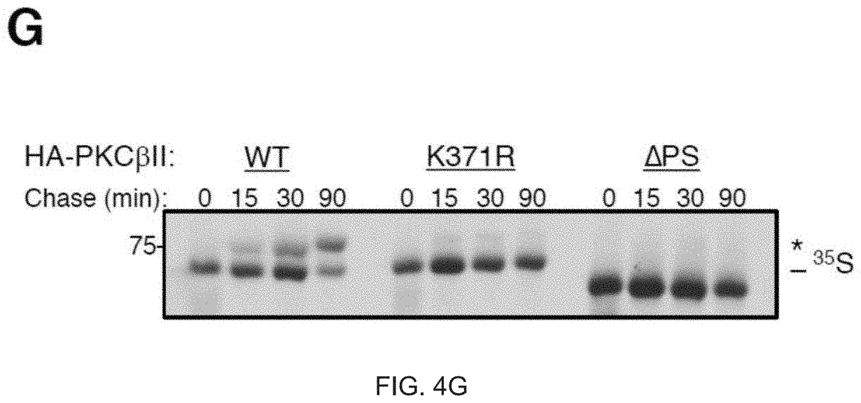

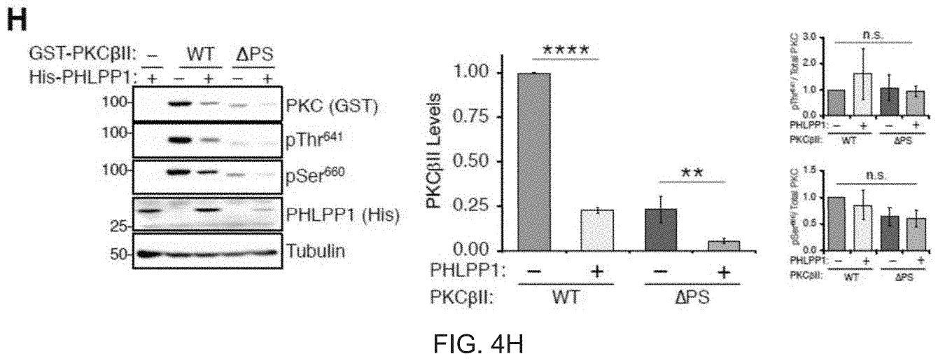

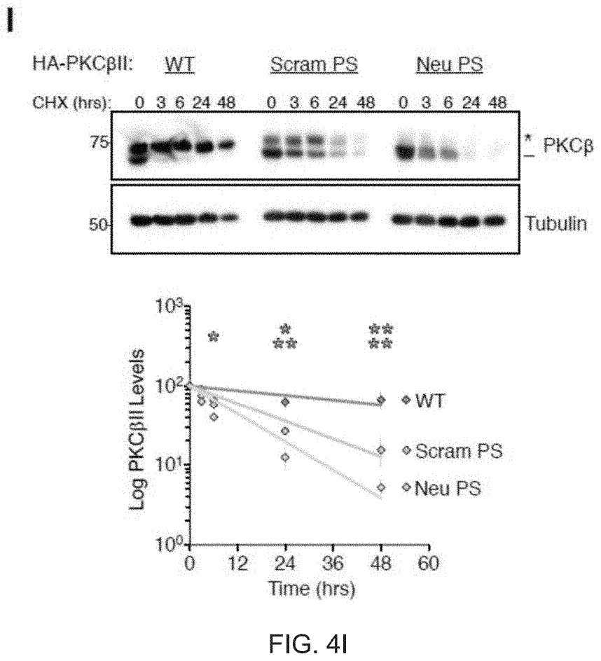

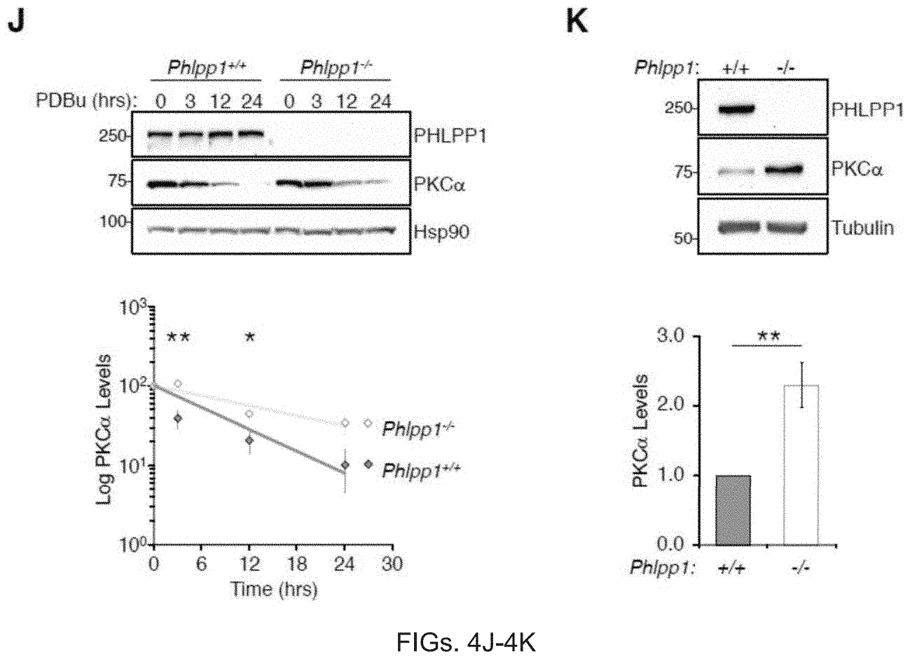

Next, whether autoinhibition-deficient PKC was subject to dephosphorylation of the exposed hydrophobic motif site was investigated. It was showed previously that activation of pure PKC via membrane binding increases its sensitivity to phosphatases by two orders of magnitude (Dutil et al., 1994). Furthermore, this phosphatase sensitivity is prevented by occupancy of the active site with protein or peptide substrates or with small-molecule inhibitors (Cameron et al., 2009; Dutil and Newton, 2000; Gould et al., 2011). To assess whether the pseudosubstrate of PKC can similarly protect the kinase domain from dephosphorylation in trans, the phosphorylation state of the isolated catalytic domain (Cat) expressed alone or co-expressed with the isolated regulatory domain containing (Reg) or lacking (Reg DPS) the pseudosubstrate was examined (FIG. 4A). The catalytic domain alone was not phosphorylated at either of the C-terminal priming sites in COS7 cells; however, co-expression with the PKC regulatory domain (Cat+Reg) was sufficient to rescue phosphorylation at the hydrophobic motif, but not the turn motif (FIG. 4B). Co-expression with the regulatory domain lacking the pseudosubstrate (Cat+Reg DPS), in contrast, did not promote catalytic domain hydrophobic motif phosphorylation (FIG. 4B). These data reveal that autoinhibition by the pseudosubstrate is responsible for retaining phosphate specifically at the hydrophobic motif.

Next, whether the known hydrophobic motif phosphatase PHLPP was responsible for the dephosphorylation of newly synthesized autoinhibition-deficient PKC was addressed. Pulse-chase analysis to 35S radiolabel a pool of newly synthesized PKC was employed and the maturation of the nascent protein was monitored via the electrophoretic mobility shift that accompanies phosphorylation (Bomer et al., 1989; Sonnenburg et al., 2001). The kinetics of PKC phosphorylation were unaffected by ectopic PHLPP1 expression (FIG. 4C), indicating that PHLPP1 may be saturating in any regulation of PKC. Immunoprecipitation revealed that PHLPP1 exclusively bound the faster-mobility, unphosphorylated species of 35S-labeled (newly synthesized) PKC and did not bind the slower-mobility band that had become phosphorylated by 60 min (FIG. 4C; asterisk). Further coimmunoprecipitation studies revealed that PHLPP1 effectively bound unphosphorylated DPS or kinase-dead (K371R) PKCβII mutants. This interaction was independent of the C1A, C1B, or C2 domains, as mutants lacking these regulatory domains still associated with PHLPP1 (FIG. 4D). Deletion of the C2 domain with the pseudosubstrate intact (AC2) also resulted in enhanced PHLPP1 association (FIG. 4D), consistent with our previous report that the C2 domain clamps the pseudosubstrate in the substrate-binding cavity (Antal et al., 2015a).

Analysis of phosphorylation state and CKAR-reported activity revealed that the PKCβII ΔC2 mutant had decreased phosphorylation and enhanced basal activity, consistent with a loosening of autoinhibition as observed upon disruption of pseudosubstrate binding (FIGS. 4E and 4F). Deletion of the C1 and C2 domains concurrently (ΔC1A/C1B/C2) resulted in greater dephosphorylation than that observed upon deletion of the C2 domain alone (ΔC2). However, PKCβII ΔC1A/C1B/C2, which retains only the pseudosubstrate segment of the regulatory domain, was effectively autoinhibited: despite its enhanced sensitivity to dephosphorylation, its basal activity was indistinguishable from that of PKCβII ΔC1A, PKCβII ΔC2, and PKCβII ΔC1A/C1B (FIG. 4F). These data reveal that the pseudosubstrate functions not only as an inhibitor of the kinase domain but also as a tether to position the regulatory domains that protect PKC from dephosphorylation.

Next, pulse-chase analysis was used to examine if phosphorylation could be detected on newly synthesized autoinhibition deficient PKC. PKCpil DPS, like kinase-dead PKC (K371R), did not undergo the characteristic mobility shift observed for WT PKC (FIG. 4G). This finding suggests that PHLPP1 dephosphorylates newly synthesized PKC that cannot be autoinhibited, thus preventing accumulation of the phosphorylated species on any “open” PKC.

Previous studies have shown that the isolated catalytic domain of PKC is phosphorylated at the priming sites when expressed in insect cells (Behn-Krappa and Newton, 1999), suggesting a different phosphatase environment in insect versus mammalian cells. To determine whether PKCpil DPS also evades dephosphorylation in this system, the phosphorylation state of WT PKCpil or PKCpil DPS expressed in Sf9 cells were analyzed. In marked contrast to its unphosphorylated state in mammalian cells, PKCpil DPS was phosphorylated at all three priming sites in Sf9 cells, revealing that PKC lacking the pseudosubstrate does incorporate phosphate but is dephosphorylated in the absence of autoinhibition in certain contexts, such as in COS7 cells (FIG. 4H). Co-expression of the PP2C phosphatase domain of PHLPP1 caused a 4-fold decrease in both PKC@ll WT and DPS steady-state levels, along with a commensurate decrease in phosphorylation, relative to cells that did not express the PHLPP1 PP2C domain (FIG. 4H). The PHLPP1-induced decrease in steady-state levels and loss of the dephosphorylated species is consistent with the dephosphorylated protein displaying enhanced sensitivity to downregulation (FIG. 4H).

Upon dephosphorylation, PKC is subject to ubiquitination and proteasome-dependent degradation (Parker et al., 1995). Analysis of PKC's half-life via cycloheximide treatment of cells confirmed that autoinhibition-deficient PKC was significantly less stable than WT PKC (FIG. 4I; WT PKCpil tin >48 h, Scram PS t112=16±2 h, Neu PS t12=10.1±0.5 h). Furthermore, endogenous PKCa was more resistant to PDBu-induced downregulation in the absence of PHLPP1 (FIG. 4J; Ph/pp14 ti/2=14±2 h, PhIpp1* t12=6.6±0.9 h). Moreover, the steady-state levels of endogenous PKCa were 2-fold higher in Ph/pp14 MEFs compared to Phlppl* MEFs (FIG. 4K). These results demonstrate that unphosphorylated PKC is unstable and support a role for PHLPP1 in regulating PKC stability by opposing hydrophobic motif phosphorylation and consequently promoting PKC degradation.

### Example 6

### Cancer-Associated Pseudosubstrate Hotspot Mutations Reveal a Distinct PKC LOF Mechanism

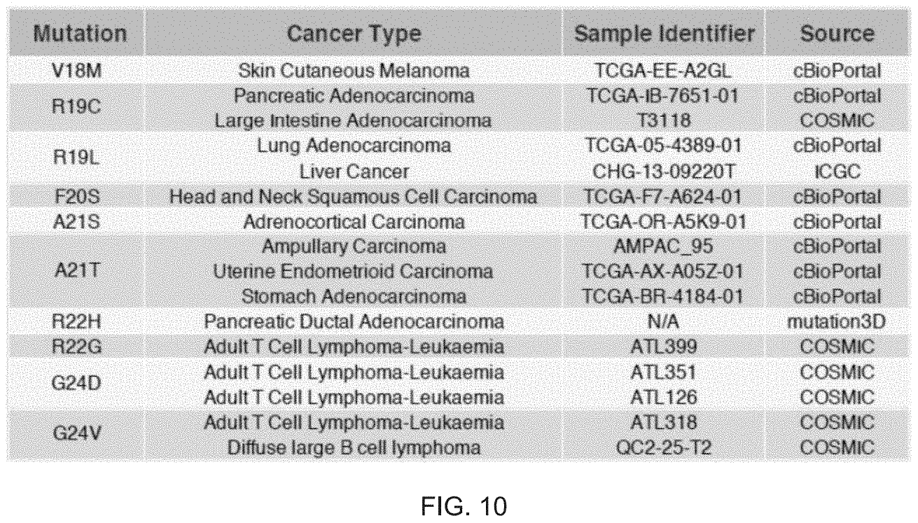

Given the tumor-suppressive role of PKCβ , whether PKC mutations that perturb autoinhibition, and are thus subject to PHLPP1 quality control, could present a LOF mechanism in cancer. In support of this, the pseudosubstrate of PKCβ is a 3D-clustered functional hotspot of cancer-associated mutations (Gao et al., 2017). 10 distinct mutations, identified in 16 tumor samples, occur in the region preceding the PO position (Ala25) of the pseudosubstrate (P-7 through P-1) (FIG. 10, FIG. 5A, SEQ ID NO: 4). These include Arg22 at the P-3 position in the pseudosubstrate, a critical residue for effective autoinhibition that makes multiple contacts with both the bound nucleotide and Asp470 in the active site (FIG. 5B) (House and Kemp, 1990; Pears et al., 1990). Analysis of sequence conservation among PKC isozymes using the protein alignment tool KinView (McSkimming et al., 2016) reveals that this interaction partner, which resides between the HRD and DFG motifs of the kinase activation segment (SEQ ID NO: 5), is highly conserved in PKC isozymes compared to other kinases (FIG. 5B, *). Given the conservation of this interaction pair, it was reasoned that the Arg22 mutations would have the largest effect on PKC autoinhibition. Introducing each of the 10 mutations into PKCβII, basal activity was measured via CKAR upon inhibitor addition in COS7 cells. The activity of every pseudosubstrate mutant differed significantly from that of WT and segregated into two distinct groups: 56% of mutations were less active than WT and 44% of mutations were more active (FIGS. 5C and 5D). Mutations displaying enhanced autoinhibition (FIG. 5E) had a higher fraction of phosphorylated PKC to unphosphorylated PKC compared to WT PKC, as assessed by the intensity of the slower-migrating phosphorylated species (asterisk) to the faster-migrating unphosphorylated species (dash) and staining with antibodies to the three processing phosphorylations (FIG. 5E). Conversely, mutations displaying reduced autoinhibition (FIG. 5E) had a lower fraction of phosphorylated PKC to unphosphorylated PKC compared to WT PKC (FIG. 5E). Thus, mutants with reduced basal activity had increased phosphorylation relative to WT and mutants with increased basal activity had reduced phosphorylation (FIG. 5F). These data show that aberrant autoinhibition of cancer- associated PKC pseudosubstrate mutations causes LOF in either of two ways: (1) enhancing pseudosubstrate affinity to reduce PKC output or (2) weakening pseudosubstrate affinity to reduce PKC phosphorylation and stability.

### Example 7

### PKC Quality Control Is Conserved in Human Cancer

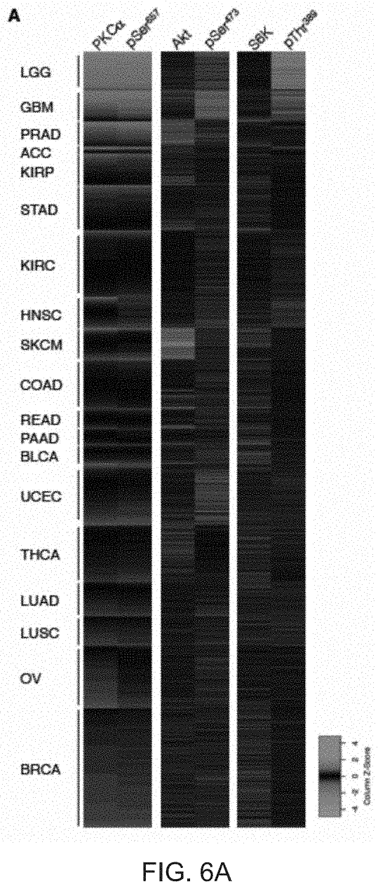

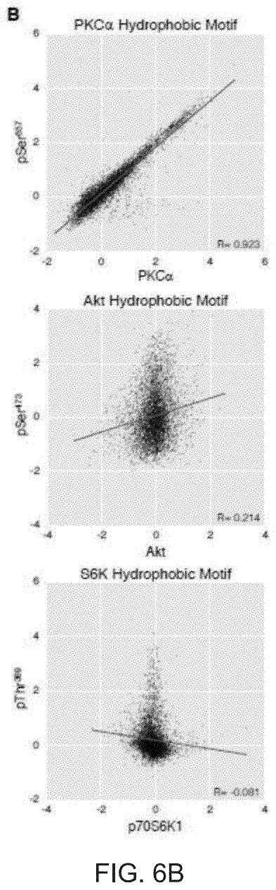

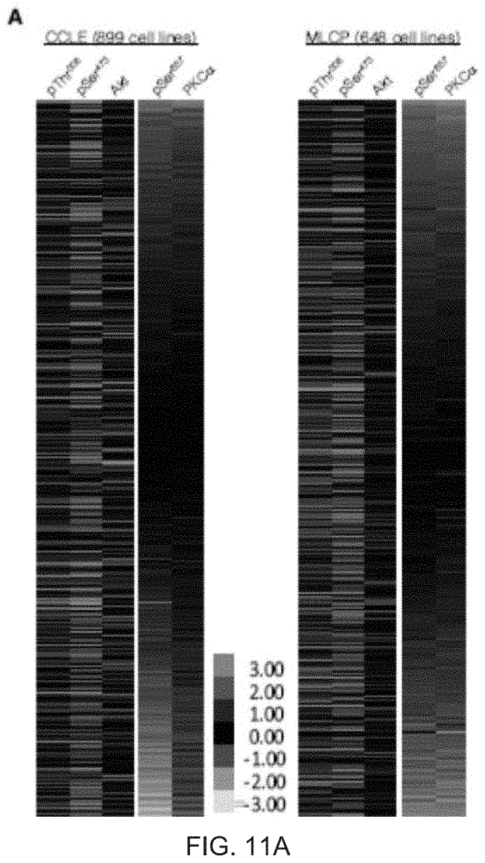

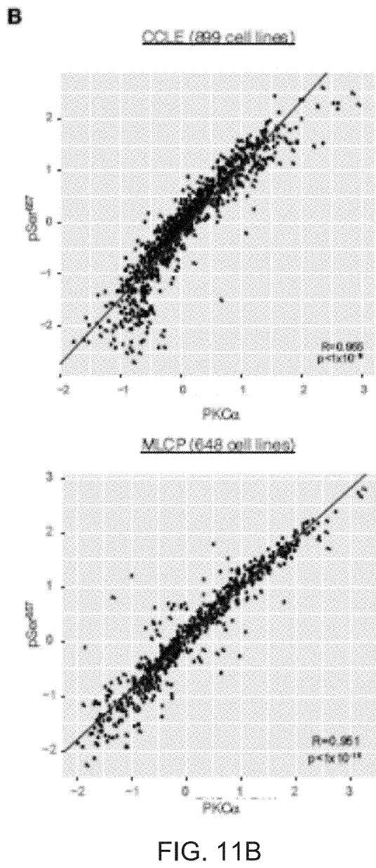

More broadly, it was explored whether PHLPP1-mediated quality control may be a ubiquitous mechanism employed by tumors to suppress PKC output. To assess whether PKC quality control by PHLPP1 is a conserved process in human cancer, the phosphorylation state and total protein levels of PKC in patient tumor samples were analyzed by reverse-phase protein array (RPPA), a high-throughput antibody-based method for quantitative detection of protein markers from cell lysates (Tibes et al., 2006). Analysis of 5,157 patient samples from 19 cancers comprising The Cancer Genome Atlas (TCGA) Pan-Can 19 revealed a striking 1:1 correlation between PKCa hydrophobic motif phosphorylation (pSer⁸⁵⁷) and total PKCa protein (FIGS. 6A and 6B; R=0.923). Hydrophobic motif phosphorylation of Akt and S6K, two other AGC kinases regulated by PHLPP1, were also analyzed. In contrast to PKC, hydrophobic motif phosphorylation of Akt (pSer4⁷³) and S6K (pThr³⁸⁹) did not correlate with total protein (FIGS. 6A and 6B; R=0.214 and −0.081, respectively). Consistent with the tumor data, analysis of cancer cell lines from the Cancer Cell Line Encyclopedia (CCLE) and MD Anderson Cell Lines Project (MCLP) also displayed a strong correlation between PKCa hydrophobic motif phosphorylation (pSer⁶⁵⁷) and total PKCa protein, which was not observed with the Akt activation loop (pThr³⁰⁸) or hydrophobic motif (pSer⁴⁷³) phosphorylation sites and total Akt protein (FIGS. 11A-11B). These data demonstrate that the hydrophobic motif site is generally phosphorylated in essentially 100% of the PKC species present in the cell, regardless of cell or tissue type. The one exception may be head and neck squamous carcinomas (HNSC), where a small number of samples had hypophosphorylated PKC; whether defects in the PKC degradation pathway allow accumulation of unphosphorylated PKC in this cancer remains to be determined.

In summary, RPPA analyses validate cellular studies showing that unphosphorylated PKC is unstable and rapidly degraded, indicating that only phosphorylated PKC accumulates in cells in an endogenous context.

### Example 8

### High PKC and Low PHLPP1 Levels Are Protective in Pancreatic Adenocarcinoma

Next, it was sought to identify which cancer subtype exhibits the most robust PKC quality control by PHLPP1, a finding that could be therapeutically relevant. It was reasoned that cancers in which PKC phosphorylation was strongly dependent on PHLPP1 would have (1) a strong correlation between PKCa and PKCβ hydrophobic motif phosphorylation due to common regulation of their levels and (2) relatively low PKC steady-state levels because of dominant regulation by PHLPP1. Thus, the correlation of total PKCa and PKCβ hydrophobic motif phosphorylation as a function of cancer type was examined (FIG. 7A; column 2).

The association of known positive regulators of PKC processing, such as PDK-1 and mTORC2 components, with the PKCa:PKCβ hydrophobic motif correlation were also examined (FIG. 7A; columns 3-7). In general, cancers with relatively high levels of PKC expression (e.g., low-grade glioma [LGG], glioblastoma multiforme [GBM], and kidney renal papillary cell carcinoma [KIRP]) had relatively high correlation with these positive regulators, and those with low levels of PKC expression had low correlation with these positive regulators. One notable outlier was pancreatic adenocarcinoma (PAAD), which showed correlation signatures with positive regulators similar to those observed in high-PKC-expressing cancers despite much lower PKCa expression levels (FIG. 7A). Thus, it is suggested that PKC expression in PAAD, which is suppressed by a common mechanism due to the strong correlation of PKCa:PKCβ hydrophobic motif phosphorylation (FIG. 7A; column 2), may be dominantly regulated by PHLPP1 quality control. Indeed, RPPA analysis of the 105 PAAD samples revealed an inverse correlation between PHLPP1 levels and PKCa levels (FIGS. 7B and 7C). In contrast, this inverse correlation between PHLPP1 levels and PKCa was not observed in the glioma tumor samples (FIG. 7B), two cancers in which positive regulators dominate in controlling PKC levels. This suggests that in gliomas, which generally have very low levels of PHLPP1 (Warfel et al., 2011), positive regulators dominate in controlling PKC levels. However, in pancreatic cancer, PKC levels are determined by the negative regulator PHLPP1 via PKC quality control, as evidenced by the inverse correlation between PHLPP1 and PKCa protein levels. Together, these findings reveal a consistent 1:1 stoichiometry of phosphorylated PKC and total PKC protein levels regardless of cell or tumor type; in some malignancies, such as pancreatic cancer, the amount of PHLPP1 is the dominant mechanism controlling PKC levels.

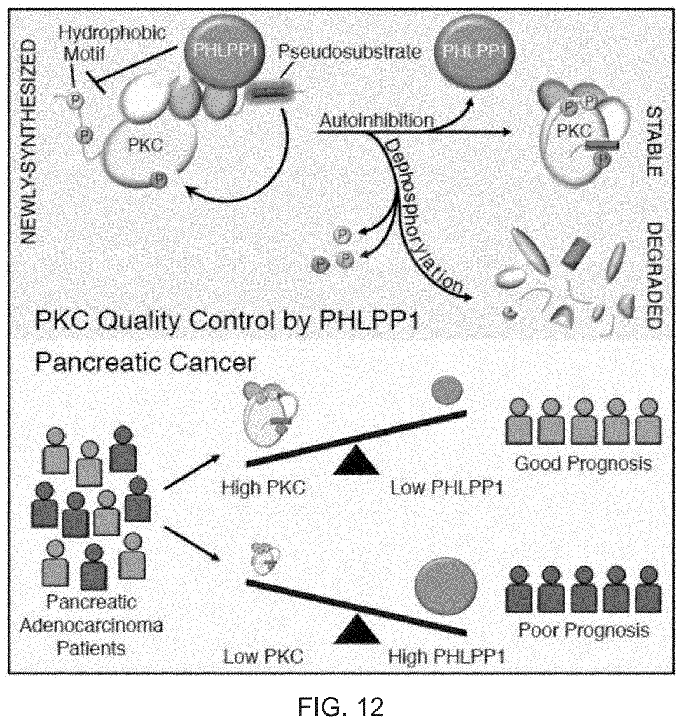

Next, the impact of PKC expression on patient outcome by stratifying survival rates by levels of PKC hydrophobic motif phosphorylation was measured. It was focused on pancreatic cancer as PKC levels are subject to PHLPP1-mediated quality control in this cancer. Analysis of the cohort of 105 PAAD patients revealed that high levels of hydrophobic motif phosphorylation in PKCa (pSer⁸⁵⁷) or PKCβ (pSer⁸O8) co-segregated with significantly improved survival (FIG. 7D). Thus, as PKC hydrophobic motif phosphorylation demarcates stable PKC and improved patient survival (see FIG. 12), it serves as a potential prognostic marker and avenue for intervention in pancreatic cancer, for which there are limited effective therapeutic options.

### REFERENCES

Alessi, D.R., Andjelkovic, M., Caudwell, B., Cron, P., Morrice, N., Cohen, P., and Hemmings, B.A. (1996). Mechanism of activation of protein kinase B by insulin and IGF-1. Embo J.Embo J 15, 6541-6551.
Almoguera, C., Shibata, D., Forrester, K., Martin, J., Amheim, N., and Perucho, M. (1988). Most human carcinomas of the exocrine pancreas contain mutant c-K-ras genes. Cell 53, 549-554.
Antal, C.E., Callender, J.A., Komev, A.P., Taylor, S. S., and Newton, A.C. (2015a). Intramolecular C2 Domain-Mediated Autoinhibition of Protein Kinase C p11. CellReports 12,1252-1260.
Antal, C.E., Hudson, A.M., Kang, E., Zanca, C., Wirth, C., Stephenson, N.L., Trotter, E.W., Gallegos, L.L., Miller, C.J., Fumari, F. B., et al. (2015b). Cancer-associated protein kinase C mutations reveal kinase's role as tumor suppressor. Cell 160, 489-502.
Antal, C.E., Violin, J.D., Kunkel, M.T., Skovso, S., and Newton, A.C. (2014). Intramolecular conformational changes optimize protein kinase C signaling. Chem Biol 21, 459-469.
Balendran, A., Hare, G.R., Kieloch, A., Williams, M. R., and Alessi, D.R. (2000). Further evidence that 3-phosphoinositide-dependent protein kinase-1 (PDK1) is required for the stability and phosphorylation of protein kinase C (PKC) isoforms. FEBS letters 484, 217-223.
Barcelo, C., Paco, N., Morell, M., Alvarez-Moya, B., Bota-Rabassedas, N., Jaumot, M., Vilardell, F., Capella, G., and Agell, N. (2014). Phosphorylation at Ser-181 of oncogenic KRAS is required for tumor growth. Cancer research 74, 1190-1199.
Bayliss, R., Haq, T., and Yeoh, S. (2015). The Ys and wherefores of protein kinase autoinhibition *. BBA - Proteins and Proteomics 1854,1586-1594.
Behn-Krappa, A., and Newton, A.C. (1999). The hydrophobic phosphorylation motif of conventional protein kinase C is regulated by autophosphorylation. Current biology: CB 9, 728-737.
Bivona, T.G., Quatela, S.E., Bodemann, B.O., Aheam, I.M., Soskis, M.J., Mor, A., Miura, J., Wiener, H.H., Wight, L., Saba, S. G., et al. (2006). PKC regulates a famesyl-electrostatic switch on K-Ras that promotes its association with Bcl-XL on mitochondria and induces apoptosis. Mol Cell 21, 481-493.
Bomancin, F., and Parker, P.J. (1997). Phosphorylation of protein kinase C-alpha on serine 657 controls the accumulation of active enzyme and contributes to its phosphatase- resistant state [published erratum appears in J Biol Chem 1997 May 16;272(20):13458]. The Journal of biological chemistry 272, 3544-3549.
Bomer, C., Filipuzzi, I., Wartmann, M., Eppenberger, U., and Fabbro, D. (1989). Biosynthesis and posttranslational modifications of protein kinase C in human breast cancer cells. The Journal of biological chemistry 264,13902-13909.
Brognard, J., and Hunter, T. (2011). Protein kinase signaling networks in cancer. Curr Opin Genet Dev 21,4-11.
Callender, J.A., Yang, Y., Lorden, G., Stephenson, N.L., Jones, A.C., Brognard, J., and Newton, A.C. (2018). Protein kinase Calpha gain-of-function variant in Alzheimer's disease displays enhanced catalysis by a mechanism that evades down-regulation. Proceedings of the National Academy of Sciences of the United States of America 115, E5497-E5505.
Cameron, A.J., Escribano, C., Saurin, A.T., Kostelecky, B., and Parker, P.J. (2009). PKC maturation is promoted by nucleotide pocket occupation independently of intrinsic kinase activity. Nature structural & molecular biology.
Cazaubon, S., Bomancin, F., and Parker, P.J. (1994). Threonine-497 is a critical site for permissive activation of protein kinase C alpha. The Biochemical journal 301, 443-448.
Chehab, N.H., Malikzay, A., Stavridi, E. S., and Halazonetis, T.D. (1999). Phosphorylation of Ser-20 mediates stabilization of human p53 in response to DNA damage. Proc Natl Acad Sci USA 96,13777-13782.
Dowling, C.M., Phelan, J., Callender, J.A., Cathcart, M.C., Mehigan, B., McCormick, P., Dalton, T., Coffey, J.C., Newton, A.C., O'Sullivan, J., et al. (2016). Protein kinase C beta II suppresses colorectal cancer by regulating IGF-1 mediated cell survival. Oncotarget 7, 20919-20933.
Dutil, E.M., Keranen, L.M., DePaoli-Roach, A. A., and Newton, A.C. (1994). In vivo regulation of protein kinase C by trans-phosphorylation followed by autophosphorylation. The Journal of biological chemistry 269, 29359-29362.
Dutil, E. M., and Newton, A.C. (2000). Dual role of pseudosubstrate in the coordinated regulation of protein kinase C by phosphorylation and diacylglycerol. The Journal of biological chemistry 275,10697-10701.
Dutil, E.M., Toker, A., and Newton, A.C. (1998). Regulation of conventional protein kinase C isozymes by phosphoinositide-dependent kinase 1 (PDK-1). Current biology: CB 8, 1366-1375.
Edwards, A. S., and Newton, A.C. (1997). Phosphorylation at conserved carboxyl-terminal hydrophobic motif regulates the catalytic and regulatory domains of protein kinase C. The Journal of biological chemistry 272,18382-18390.
Gao, J., Chang, M.T., Johnsen, H.C., Gao, S.P., Sylvester, B.E., Sumer, S.O., Zhang, H., Solit, D.B., Taylor, B.S., Schultz, N., et al. (2017). 3D clusters of somatic mutations in cancer reveal numerous rare mutations as functional targets. Genome Med 9,4.
Gao, T., Brognard, J., and Newton, A.C. (2008). The Phosphatase PHLPP Controls the Cellular Levels of Protein Kinase C. Journal of Biological Chemistry 283, 6300-6311.
Gao, T., Fumari, F., and Newton, A.C. (2005). PHLPP: a phosphatase that directly dephosphorylates Akt, promotes apoptosis, and suppresses tumor growth. Molecular cell 18, 13-24.
Gould, C.M., Antal, C.E., Reyes, G., Kunkel, M.T., Adams, R.A., Ziyar, A., Riveros, T., and Newton, A.C. (2011). Active site inhibitors protect protein kinase C from dephosphorylation and stabilize its mature form. The Journal of biological chemistry 286, 28922-28930.
Griner, E. M., and Kazanietz, M.G. (2007). Protein kinase C and other diacylglycerol effectors in cancer. Nat Rev Cancer 7, 281-294.
Guertin, D.A., Stevens, D.M., Thoreen, C.C., Burds, A.A., Kalaany, N.Y., Moffat, J., Brown, M., Fitzgerald, K. J., and Sabatini, D.M. (2006). Ablation in mice of the mTORC components raptor, rictor, or mLST8 reveals that mTORC2 is required for signaling to Akt-FOXO and PKCalpha, but not S6K1. Dev Cell 11, 859-871.
Hansra, G., Garcia-Paramio, P., Prevostel, C., Whelan, R.D., Bomancin, F., and Parker, P.J. (1999). Multisite dephosphorylation and desensitization of conventional protein kinase C isotypes. Biochem J 342 (Pt 2), 337-344.
House, C., and Kemp, B.E. (1987). Protein kinase C contains a pseudosubstrate prototope in its regulatory domain. Science 238, 1726-1728.
House, C., and Kemp, B.E. (1990). Protein kinase C pseudosubstrate prototope: structure-function relationships. Cell Signal 2, 187-190.
Hsu, A.H., Lum, M.A., Shim, K.-S., Frederick, P.J., Morrison, C.D., Chen, B., Lele, S.M., Sheinin, Y.M., Daikoku, T., and Dey, S.K. (2018). Crosstalk between PKCa and P13K/AKT Signaling Is Tumor Suppressive in the Endometrium. Cell reports 24, 655-669.
Huang, L.C., Ross, K.E., Baffi, T.R., Drabkin, H., Kochut, K. J., Ruan, Z., D'Eustachio, P., McSkimming, D., Arighi, C., Chen, C., et al. (2018). Integrative annotation and knowledge discovery of kinase post-translational modifications and cancer-associated mutations through federated protein ontologies and resources. Sci Rep 8, 6518.
Jaken, S., Tashjian, A. H., Jr., and Blumberg, P. M. (1981). Characterization of phorbol ester receptors and their down-modulation in GH4C1 rat pituitary cells. Cancer research 41, 2175-2181.
Keranen, L. M., Dutil, E. M., and Newton, A.C. (1995). Protein kinase C is regulated in vivo by three functionally distinct phosphorylations. Current Biology 5, 1394-1403.
Le Good, J. A., Ziegler, W. H., Parekh, D. B., Alessi, D. R., Cohen, P., and Parker, P.J. (1998). Protein kinase C isotypes controlled by phosphoinositide 3-kinase through the protein kinase PDK1. Science 281, 2042-2045.
Li, J., Zhao, W., Akbani, R., Liu, W., Ju, Z., Ling, S., Vellano, C. P., Roebuck, P., Yu, Q., Eterovic, A. K., et al. (2017). Characterization of Human Cancer Cell Lines by Reverse-phase Protein Arrays. Cancer Cell 31, 225-239.
Liu, J., Stevens, P. D., Li, X., Schmidt, M. D., and Gao, T. (2011). PHLPP-mediated dephosphorylation of S6K1 inhibits protein translation and cell growth. Molecular and cellular biology.
Lu, Z., Liu, D., Homia, A., Devonish, W., Pagano, M., and Foster, D.A. (1998). Activation of protein kinase C triggers its ubiquitination and degradation. Molecular and cellular biology 18, 839-845.
Masubuchi, S., Gao, T., O'Neill, A., Eckel-Mahan, K., Newton, A.C., and Sassone-Corsi, P. (2010). Protein phosphatase PHLPP1 controls the light-induced resetting of the circadian clock. Proceedings of the National Academy of Sciences 107,1642-1647.
McSkimming, D. I., Dastgheib, S., Baffi, T.R., Byrne, D. P., Ferries, S., Scott, S. T., Newton, A.C., Eyers, C.E., Kochut, K. J., Eyers, P. A., et al. (2016). KinView: a visual comparative sequence analysis tool for integrated kinome research. Molecular BioSystems 12, 3651-3665.
Milani, G., Rebora, P., Accordi, B., Galla, L., Bresolin, S., Cazzaniga, G., Buldini, B., Mura, R., Ladogana, S., Giraldi, E., et al. (2014). Low PKCalpha expression within the MRD-HR stratum defines a new subgroup of childhood T-ALL with very poor outcome. Oncotarget 5, 5234-5245.
Newton, A.C. (2018). Protein kinase C: perfectly balanced. Crit Rev Biochem Mol Biol 53, 208-230.
Orr, J. W., Keranen, L. M., and Newton, A.C. (1992). Reversible exposure of the pseudosubstrate domain of protein kinase C by phosphatidylserine and diacylglycerol. The Journal of biological chemistry 267,15263-15266.
Orr, J. W., and Newton, A.C. (1994). Requirement for Negative Charge on “Activation Loop” of Protein Kinase C. J. Biol. Chem. 269, 27715-27718.
Parikh, C., Janakiraman, V., Wu, W. I., Foo, C. K., Kljavin, N. M., Chaudhuri, S., Stawiski, E., Lee, B., Lin, J., Li, H., et al. (2012). Disruption of PH-kinase domain interactions leads to oncogenic activation of AKT in human cancers. Proc Natl Acad Sci USA 109,19368-19373.
Park, W. S., Heo, W. D., Whalen, J. H., O'Rourke, N. A., Bryan, H. M., Meyer, T., and Teruel, M. N. (2008). Comprehensive identification of PIP3-regulated PH domains from C. elegans to H. sapiens by model prediction and live imaging. Mol Cell 30, 381-392.
Parker, P.J., Bosca, L., Dekker, L., Goode, N. T., Hajibagheri, N., and Hansra, G. (1995). Protein kinase C (PKC)-induced PKC degradation: a model for down-regulation. Biochemical Society transactions 23, 153-155.
Pears, C. J., Kour, G., House, C., Kemp, B. E., and Parker, P.J. (1990). Mutagenesis of the pseudosubstrate site of protein kinase C leads to activation. European journal of biochemistry 194, 89-94.
Scott, A. M., Antal, C. E., and Newton, A.C. (2013). Electrostatic and hydrophobic interactions differentially tune membrane binding kinetics of the C2 domain of protein kinase Calpha. J Biol Chem 288,16905-16915.
Sonnenburg, E. D., Gao, T., and Newton, A.C. (2001). The phosphoinositide-dependent kinase, PDK-1, phosphorylates conventional protein kinase C isozymes by a mechanism that is independent of phosphoinositide 3-kinase. The Journal of biological chemistry 276, 45289-45297.
Taylor, S. S., and Komev, A. P. (2011). Protein kinases: evolution of dynamic regulatory proteins. Trends Biochem Sci 36, 65-77.
Tibes, R., Qiu, Y., Lu, Y., Hennessy, B., Andreeff, M., Mills, G. B., and Komblau, S.M. (2006). Reverse phase protein array: validation of a novel proteomic technology and utility for analysis of primary leukemia specimens and hematopoietic stem cells. Mol Cancer Ther 5, 2512-2521.
Violin, J. D., Zhang, J., Tsien, R. Y., and Newton, A.C. (2003). A genetically encoded fluorescent reporter reveals oscillatory phosphorylation by protein kinase C. The Journal of cell biology 161, 899-909.
Wang, M.T., Holderfield, M., Galeas, J., Delrosario, R., To, M. D., Balmain, A., and McCormick, F. (2015). K-Ras Promotes Tumorigenicity through Suppression of Non-canonical Wht Signaling. Cell 163,1237-1251.
Warfel, N. A., Niederst, M., Stevens, M. W., Brennan, P. M., Frame, M. C., and Newton, A.C. (2011). Mislocalization of the E3 ligase, beta-transducin repeat-containing protein 1 (beta-TrCP1), in glioblastoma uncouples negative feedback between the pleckstrin homology domain leucine-rich repeat protein phosphatase 1 (PHLPP1) and Akt. J Biol Chem 286,19777-19788.
Weinberg, R.A. (1991). Tumor suppressor genes. Science 254,1138-1146.
Zhang, L. L., Cao, F. F., Wang, Y., Meng, F. L., Zhang, Y., Zhong, D. S., and Zhou, Q. H. (2015). The protein kinase C (PKC) inhibitors combined with chemotherapy in the treatment of advanced non-small cell lung cancer: meta-analysis of randomized controlled trials. Clinical & translational oncology: official publication of the Federation of Spanish Oncology Societies and of the National Cancer Institute of Mexico 17, 371-377.

The preceding disclosure and/or examples are offered for illustrative purposes only and are not intended to limit the scope of the invention in any way. Various modifications of the invention in addition to those shown and described herein will become apparent to those skilled in the art from the foregoing description and fall within the scope of the appended claims.

1. A method for diagnosing or prognosing a cancer in a subject, the method comprising:
(a) obtaining a sample from the subject;
(b) measuring a level of at least one biomarker in the subject; and
(c) comparing the level of the at least one biomarker in the subject to a level of the at least one biomarker in a control sample;
wherein the control sample is from a subject who does not have cancer; and
wherein the at least one biomarker is selected from protein kinase C (PKC), PH domain and leucine rich repeat protein phosphatase 1 (PHLPP1), or a ratio of PKC/PHLPP1.

2. The method of claim 1, wherein the at least one biomarker is PKC and the level of the at least one biomarker is higher than the level in the control sample.

3. The method of claim 2, wherein a higher level of PKC in the sample from the patient than the level of PKC in the control sample is associated with an increased cancer survival rate.

4. The method of claim 2, wherein a lower level of PKC in the sample from the patient than the level of PKC in the control sample is associated with a decreased cancer survival rate.

5. The method of claim 1, wherein the at least one biomarker is PHLPP1 and the level of the at least one biomarker is lower than the level in the control sample.

6. The method of claim 5, wherein a lower level of PHLPP1 in the sample from the patient than the level of PHLPP1 in the control sample is associated with an increased cancer survival rate.

7. The method of claim 5, wherein a higher level of PHLPP1 in the sample from the patient than the level of PHLPP1 in the control sample is associated with a decreased cancer survival rate.

8. The method of claim 1, wherein the at least one biomarker is the ratio of PKC/PHLPP1 and the level of the at least one biomarker is higher than the level in the control sample.

9. The method of claim 8, wherein the ratio of PKC/PHLPP1 is greater than 1:1.

10. The method of claim 8, wherein a higher level of the ratio of PKC/PHLPP1 in the sample from the patient than the level of the ratio of PKC/PHLPP1 in the control sample is associated with an increased cancer survival rate.

11. The method of claim 8, wherein the ratio of PKC/PHLPP1 is lower than 1:1.

12. The method of claim 8, wherein a lower level of the ratio of PKC/PHLPP1 in the sample from the patient than the level of the ratio of PKC/PHLPP1 in the control sample is associated with a decreased cancer survival rate.

13. The method of claim 1, wherein the cancer comprises pancreatic adenocarcinoma, colon cancer, breast cancer, ovarian cancer, Wilms tumor, prostate cancer, hepatocellular carcinoma, glioblastoma multiforme, kidney renal papillary cell carcinoma, chronic myelogenous leukemia, non-small cell lung cancer, diffuse large B-cell lymphoma, chronic lymphocytic leukemia, renal cell carcinoma, bladder cancer, melanoma, low grade glioma, or any combination thereof.

14. The method of claim 1, wherein the cancer is pancreatic adenocarcinoma.

15. The method of claim 1, wherein the sample comprises whole blood, serum, plasma, a fine needle biopsy sample from a tumor, a fine needle aspirate sample from a tumor, a core needle biopsy sample from a tumor, an excisional biopsy sample, or any combination thereof.

16. The method of claim 1 wherein the at least one biomarker is PKC and the level of the at least one biomarker is measured by the method comprising:
(a) contacting the sample with an anti-PKC antibody;
(b) determining an amount of antibody binding using an antibody quantification technique; and
(c) correlating the amount of antibody binding to the level of PKC in the sample.

17. The method of claim 1 wherein the at least one biomarker is PHLPP1 and the level of the at least one biomarker is measured by the method comprising:
(a) contacting the sample with an anti-PHLPP1 antibody;
(b) determining an amount of antibody binding using an antibody quantification technique; and
(c) correlating the amount of antibody binding to the level of PHLPP1 in the sample.

18. The method of claim 1 wherein the at least one biomarker is a ratio of PKC/PHLPP1 and the level of the at least one biomarker is measured by the method comprising:
(a) contacting the sample with an anti-PKC antibody;
(b) contacting the sample with an anti-PHLPP1 antibody;
(c) determining an amount of anti-PKC antibody binding using an antibody quantification technique;
(d) correlating the amount of antibody binding to the level of PKC in the sample;
(e) determining an amount of anti-PHLP1 antibody binding using an antibody quantification technique;
(f) correlating the amount of antibody binding to the level of PHLPP1 in the sample; and
(g) calculating a ratio of PKC/PHLPP1.

19. The method of claim 16,
wherein the antibody quantification technique comprises immunofluorescence, radiolabeling, immunoblotting, Western blotting, enzyme-linked immunosorbent assay, flow cytometry, immunoprecipitation, immunohistochemistry, biofilm test, affinity ring test, antibody array optical density test, chemiluminescence, or any combination thereof.

20. A method for treating a cancer in a subject, the method comprising:
(a) obtaining a sample from the subject;
(b) measuring a level of at least one biomarker in the subject;
(c) comparing the level of the at least one biomarker in the subject to a level of the at least one biomarker in a control sample; and
(d) administering an effective amount of a PHLPP1 inhibitor to the subject;
wherein the control sample is from a subject who does not have cancer; and
wherein the at least one biomarker is selected from protein kinase C (PKC), PH domain and leucine rich repeat protein phosphatase 1 (PHLPP1), or a ratio of PKC/PHLPP1.

21. The method of claim 20, wherein the PHLPP1 inhibitor comprises NSC117079, NSC45586, or any combination thereof.

22. The method of claim 20, wherein the cancer is pancreatic adenocarcinoma.


SEQUENCE LISTING (5 sequences)

SEQ ID NO: 1 - protein, 13 aa, Homo sapiens
VRFARKGALRQKN

SEQ ID NO: 2 - protein, 13 aa, Artificial sequence
  Synthetic construct
NRARGRAFKVKAL

SEQ ID NO: 3 - protein, 13 aa, Artificial sequence
  Synthetic construct
VLFAAAGALLQNN

SEQ ID NO: 4 - protein, 7 aa, Homo sapiens
VRFARKG

SEQ ID NO: 5 - protein, 14 aa, Homo sapiens
GIIYRDLKLDNVLL